(19) United States
(12) Patent Application Publication
(10) Pub. No.: US 2025/0000610 A1
(43) Pub. Date: Jan. 2, 2025

(54) CLEANING DEVICES AND SYSTEMS FOR SURGICAL INSTRUMENTS, AND METHODS THEREOF

(71) Applicant: Bayou Surgical, Inc., Houston, TX (US)

(72) Inventors: Charles HOUSSIERE, Houston, TX (US); Joshua CORSO, Houston, TX (US); Scott SLOSS, Houston, TX (US); Rebecca SMITH, Houston, TX (US); Ashok GOWDA, Bellaire, TX (US); Abdul UMARU, Houston, TX (US); Jorge SALAZAR, Houston, TX (US); William E. COHN, Bellaire, TX (US); Rajitha ALURU, Houston, TX (US)

(21) Appl. No.: 18/885,519

(22) Filed: Sep. 13, 2024

Related U.S. Application Data
(63) Continuation of application No. PCT/US2023/064448, filed on Mar. 15, 2023.
(60) Provisional application No. 63/320,018, filed on Mar. 15, 2022.
(60) Provisional application No. 63/320,023, filed on Mar. 15, 2022.

(51) Int. Cl. A61B 90/70; A61L 2/18; A61L 2/20

(52) U.S. Cl. CPC A61B 90/70; A61L 2/18; A61L 2/20; A61B 2090/701

(57) ABSTRACT

Embodiments described herein relate to systems, devices, and methods of cleaning endoscopes or other instruments during surgical procedures. In some embodiments, a system or device can include a trocar including a trocar shaft and a cap that collectively define fluid and/or electrical passages. In some embodiments, a system or device can include obturators with a wiping element and/or an absorbent element configured to remove debris and moisture from the interior walls of trocars. In some embodiments, a system or device can include a trocar with a liquid and gas interconnect device and venting systems for cleaning instruments disposed in the trocar channel.

### CROSS-REFERENCE TO RELATED APPLICATIONS

This application is a continuation of International Patent Application No. PCT/US2023/064448, filed Mar. 15, 2023, titled “CLEANING DEVICES AND SYSTEMS FOR SURGICAL INSTRUMENTS, AND METHODS THEREOF,” which claims priority to U.S. Provisional Patent Application No. 63/320,018, filed Mar. 15, 2022, titled “CLEANING DEVICES AND SYSTEMS FOR SURGICAL INSTRUMENTS INCLUDING GAS AND LIQUID DELIVERY AND VENTING,” and U.S. Provisional Patent Application No. 63/320,023, filed Mar. 15, 2022, titled “CLEANING DEVICES AND SYSTEMS FOR SURGICAL INSTRUMENTS INCLUDING TROCARS WITH SENSING AND FLUID DELIVERY,” the disclosures of which are hereby incorporated by reference in their entirety.

### TECHNICAL FIELD

Embodiments described herein relate to systems, devices, and methods for cleaning instruments, including trocars for cleaning endoscopes having fluid delivery and sensing functions, liquid and gas interconnections, and venting elements.

### BACKGROUND

Use of small cameras in patient anatomy (e.g., body lumens or cavities) can aid in viewing interiors of the lumens while performing surgical procedures on or within the lumen or cavities. One common example of a camera is an endoscope. An endoscope is a medical device utilized for medical procedures requiring the visualization of internal organs in diagnostic or surgical settings generally referred to as a minimally invasive procedure. A physician may utilize an endoscope to make a diagnosis and/or to gain access to internal organs for treatment. The endoscope may be introduced into a patient's body via a natural orifice or through a small surgical incision.

Endoscopes and other cameras are used with an imaging system and an illumination system. The imaging system receives image data captured by the endoscope, and the illumination system can be used to provide light to assist in image capture. The three systems work together to give the physician clear views. During use, however, the tip of an endoscope or other camera may become obscured, e.g., due to smears, residue, debris, condensation, and/or other types of obstructing material. Therefore, there exists a need for an efficient way to clean the end of endoscopes and other cameras during a surgical operation.

### SUMMARY

Embodiments described herein relate to a trocar designed to clean the end of an imaging device, such as, for example, an endoscope. In some embodiments, the trocar can be positioned in a body cavity (e.g., thoracic cavity, abdominal cavity). In laparoscopic surgery, for example, the trocar penetrates through the thickness of the abdominal or chest wall to provide access for various instruments to the interior of the body. The trocar is designed such that instruments can be inserted into the body interior without the significant loss of insufflation gases used to expand the body cavity. As such, the trocar is configured to create and maintain a working space for those instruments. In some embodiments, an endoscope can be placed through the trocar, and an imaging system receiving signals from the endoscope can display images of the body interior to guide the surgical procedure.

In some embodiments, the trocar can be configured to deliver quantities of liquid and/or gas to clean the end of the endoscope. In some embodiments, a connector or cable for delivering gas and/or liquid to a trocar can include components for coupling a liquid flow to a gas flow. In some embodiments, the connector can include a liquid/gas interconnect that combines a high-flow gas lumen to a small-bore diameter liquid lumen in a ‘T-shaped’ configuration with a single output. The pressurized gas that flows through the high-flow gas lumen can propel a small bolus of liquid solution and create a transient high-energy spray to wash an obscured end of an endoscope in a controlled sequence. One-way valves are fitted to the gas lumen and the liquid lumen to prevent cross-contamination between the gas and liquid lines during normal use of the system and failure conditions. The liquid lumen geometry can minimize trapped air, accommodate one-way valve compliance, and/or prevent the liquid solution from being drawn into the gas lumen. The gas lumen geometry can allow the system to be purged without impact on the liquid and can prevent regurgitation of the solution upstream into the gas line.

In some embodiments, the trocar can include one or more sensors. For example, the trocar can include a sensor for sensing the position of the distal end of an endoscope for cleaning. In some embodiments, the trocar can include elements for venting gases from within a body cavity. In some embodiments, a venting device can include a valve and/or a filter. In some embodiments, the venting device can be integrated into an insufflation line (e.g., using a stopcock).

In some embodiments, systems, devices, and methods described herein can include an obturator that can be used with the trocar for insertion of the trocar within a body cavity. The obturator can include a wiping clement and/or an absorbent element configured to remove debris and moisture from the interior walls of trocars. In some embodiments, the trocar and the obturator can have an asymmetrical shape, such that the obturator fits into the trocar in a specific manner such that the distal surfaces of the trocar and the obturator form continuous services for penetrating through body tissue. Methods for using cleaning systems can include delivery of liquid and/or gas for cleaning (e.g., timed to sensed position/orientation), for priming, and/or for fogging mitigation.

In some embodiments, an apparatus includes: a shaft defining a channel for receiving an instrument, the shaft having a distal end that is disposable within patient anatomy; an interconnector configured to couple to a liquid source and a gas source, the interconnector including a first valve configured to control delivery of the liquid and a second valve configured to control delivery of the gas, the interconnector configured to combine separate volumes of the liquid and the gas into a combined volume of the liquid and the gas; an ejection port disposed near a distal end of the shaft, the ejection port fluidically coupled to the interconnector and configured to eject the combined volume of the liquid and the gas into the channel in response to a distal end of the instrument being disposed near the ejection port; and a sensor disposed near the ejection port, the sensor configured to detect when the distal end of the instrument is disposed near the ejection port.

In some embodiments, an apparatus includes: a shaft defining a channel for receiving an instrument, the shaft having a distal end that is disposable within patient anatomy; a cap couplable to the shaft, the cap including a fluid port configured to couple to a liquid source and a gas source and an electrical port configured to couple to a controller; an ejection port disposed near a distal end of the shaft, the ejection port configured to eject a predetermined volume of liquid and gas into the channel in response to a distal end of the instrument being disposed near the ejection port; and a fluid passage defined by at least one of the shaft or the cap and extending along a longitudinal length of the shaft, the fluid passage configured to convey the predetermined volume of liquid from the fluid port to the ejection port; a sensor configured to detect when the distal end of the instrument is disposed near the ejection port; and an electrical line disposed in at least one of the shaft or the cap and extending along the longitudinal length of the shaft, the electrical line configured to couple the electrical port to the sensor.

In some embodiments, an apparatus includes: a shaft defining a channel for receiving an instrument, the shaft having a distal end that is disposable within patient anatomy; an ejection port disposed near a distal end of the shaft, the ejection port configured to eject a predetermined volume of liquid and gas into the channel in response to a distal end of the instrument being disposed near the ejection port; a sensor disposed near the ejection port, the sensor configured to detect when the distal end of the instrument is disposed near the ejection port; a vent fluidically coupled to the channel and configured to vent fluids from within the patient anatomy to an exterior of the patient anatomy; and a filter disposed along a pathway of the vent and being configured to filter the fluids being vented through the vent.

In some embodiments, an apparatus includes: a shaft defining a channel for receiving an instrument, the shaft having a distal end that is disposable within patient anatomy, the channel having an asymmetrical cross-section defined by a plurality of side walls where a first side wall of the plurality of side walls extends out further than one or more remaining side walls of the plurality of side walls; an ejection port disposed near a distal end of the shaft, the ejection port configured to eject a predetermined volume of liquid and gas into the channel in response to a distal end of the instrument being disposed near the ejection port, the ejection port being located in the channel on the first side wall; and a sensor disposed near the ejection port, the sensor configured to detect when the distal end of the instrument is disposed near the ejection port.

In some embodiments, a system includes: an elongate device having a distal end that is disposable within patient anatomy, the shaft including: a channel for receiving an instrument; an ejection port configured to eject a volume of liquid and gas into the channel in response to a distal end of the instrument being disposed near the ejection port; and a sensor configured to detect when the instrument is disposed near the ejection port; a controller operatively coupled to the elongate device, the controller configured to receive one or more signals from the sensors and to control delivery of the liquid and the gas to the elongate device; a connector including a plurality of lines configured to couple the elongate device to the controller, a liquid source, and a gas source; and at least one filter disposed along one or more of the plurality of lines, the at least one filter configured to filter at least one of the liquid or the gas prior to being delivered to the elongate device.

In some embodiments, a method includes: detecting that an imaging device has been retracted a predetermined distance into a channel of a trocar disposed within patient anatomy; in response to the detecting, activating a delivery of pressurized gas to propel and deliver a predetermined volume of liquid to the trocar; and delivering, via an ejection port disposed in the trocar, the predetermined volume of liquid into the channel of the trocar to clean a distal end of the imaging device.

In some embodiments, a method includes: detecting that an imaging device has been retracted a predetermined distance into a channel of a trocar disposed within patient anatomy; in response to the detecting, activating a delivery of pressurized gas through an interconnect configured to combine separate volumes of the gas and a liquid into a combined volume of gas and liquid; and delivering, via an ejection port, the combined volume of gas and liquid into the channel of the trocar to clean a distal end of the imaging device.

### BRIEF DESCRIPTION OF THE DRAWINGS

The skilled artisan will understand that the drawings primarily are for illustrative purposes and are not intended to limit the scope of the inventive subject matter described herein. The drawings are not necessarily to scale; in some instances, various aspects of the inventive subject matter disclosed herein may be shown exaggerated or enlarged in the drawings to facilitate an understanding of different features. In the drawings, like reference characters generally refer to like features (e.g., functionally similar and/or structurally similar elements). Also in the drawings, optional items are shown in dashed lines.

FIG. 1 is a block diagram of a cleaning system for cleaning an endoscope disposed in a patient's anatomy (e.g., body lumen or cavity), according to an embodiment.

FIG. 2 is a block diagram of a fluid delivery system of a cleaning system, according to an embodiment.

FIG. 3 is a diagram of a trocar of a cleaning system, according to an embodiment.

FIG. 4 is a diagram of a trocar of a cleaning system with an attachable cap, according to an embodiment.

FIG. 5 is a diagram of an obturator that can be used with a trocar of a cleaning system, according to an embodiment.

FIGS. 6A-6B are cross-sectional views of a trocar shaft of a cleaning system, according to an embodiment.

FIG. 7 is a cross sectional view of a trocar lumen of a cleaning system, according to an embodiment.

FIGS. 8A-8O are illustrations of a trocar and an obturator that can be used with a cleaning system, and various components thereof, according to an embodiment.

FIGS. 9A-9C are illustrations of a stopcock switch that can be used with a cleaning system, according to an embodiment.

FIGS. 10A-10D are illustrations of power and fluidic connections to a trocar in a cleaning system, according to an embodiment.

FIG. 11 illustrates insertion of an obturator with a cleaning element into a trocar of a cleaning system, according to an embodiment.

FIGS. 12A-12C are illustrations of connections between components of a cleaning system and the relative positioning of such components and an endoscope for cleaning the endoscope, according to an embodiment.

FIGS. 13A-13B illustrate methods associated with operating cleaning systems, according to various embodiments.

FIG. 14 is a diagram of a liquid/gas interconnect of a cleaning system, according to an embodiment.

FIG. 15 is a detailed diagram of a liquid/gas interconnect of a cleaning system, according to an embodiment.

FIGS. 16A-16D are illustrations of components of a filtration system of a cleaning system, according to an embodiment.

FIG. 17 illustrates the effect on fluid flow rates associated with using different filters with a cleaning system, according to embodiments.

FIG. 18 is a flow chart of a method associated with the operation of a cleaning system, according to embodiments.

### DETAILED DESCRIPTION

Embodiments described herein relate to systems, devices, and methods for cleaning imaging devices, such as, for example, endoscopes. Such systems, devices, and methods can be configured to clean the imaging devices while they are positioned within patient anatomy (e.g., a body lumen or cavity), e.g., when they are in use during a surgical procedure. In some embodiments, a cleaning system for cleaning an endoscope can be configured to use gas to propel small, controlled amounts of a liquid into a lumen of a trocar. In some embodiments, the gas can include carbon dioxide (CO₂), nitrogen, argon, or any other suitable inert gas or combinations thereof. A liquid such as a wash solution is used to clean a distal end of the imaging devices. In some embodiments, an obturator can be shaped to fit in the trocar.

The obturator and/or the trocar can be shaped for insertion of the trocar into a body lumen or cavity. The shape of the obturator can be a function of the shape of the trocar lumen so that it can be inserted in a smooth manner and not have areas that grab tissues. If the trocar and the obturator have irregular shapes, this can ensure a glove-type fit between the two. The asymmetrical shape of the trocar lumen can also create a space between the wall of the trocar and the wall of the camera when a circular endoscope is placed inside the trocar. The asymmetrical shape of the trocar lumen can also prevent the trocar lumen from being blocked by the camera body. A space can always be present between the body of the camera and the walls of the trocar lumen, such that the outer surface of the camera is not able to rest on the wall of the trocar lumen. This ensures fluids are expelled and do not build up inside the trocar. This can help prevent fluid buildup inside the trocar. During surgery, debris and moisture can collect on interior walls of a trocar. The shape of the trocar lumen can affect whether tissues blood, or moisture can collect in the lumen during cleaning. This debris and moisture can originate from a body lumen or cavity, wherein the trocar is placed. Other sources of debris and moisture can include a wash solution that is ejected to clean an endoscopic camera during surgery. Insertion of an obturator with cleaning elements can clean the walls of the trocar. Cleaning elements can include wiping elements (e.g., squeegees) and/or absorbent elements (e.g., cloth).

Endoscopes can vary significantly. Some endoscopes use optical light, while others use infrared. Different endoscopes can also have different sizes and/or configurations. For example, some endoscopes can have a flat distal end, while other endoscopes can have angled or tapered distal ends. Systems and devices described herein can include sensors (e.g., light sensors) that can be configured to detect when an endoscope passes the sensor for a variety of different endoscopes. Once an endoscope is in a position to be cleaned, a bolus of wash solution is ejected onto the endoscope. After the ejection of the wash solution, a line containing the wash solution can be primed with fluid for a subsequent wash sequence. In some embodiments described herein, sensors on the trocar can detect illumination features at the end of the endoscope, thus identifying the precise position of the end of the endoscope, relative to the trocar shaft. The optical window of the endoscope that receives the image is nearly always in close proximity to the illumination features, and therefore, systems as described herein can detect the location of the optical end of the scope that has become soiled by surgical debris. Precisely positioned fluid and gas outlets on the inside of the trocar can then be actuated to provide a precise cleaning burst onto the tip of the endoscope, once the position of the endoscope has been detected by the optical sensors that locate the position of the illumination features.

In addition, some embodiments of cleaning systems described herein can include filters and/or vents on the trocar devices. In some embodiments, a filter can be used to filter liquids that are being delivered into the body cavity. In some embodiments, a vent can be used to vent excess gases and/or liquids from within a body lumen or cavity, e.g., to prevent overpressure. The vent can optionally include filters for filtering the vented gases and/or liquids, e.g., to reduce noxious and/or odorous fumes.

Examples of endoscope cleaning systems are described in U.S. Patent Publication No. 2019/0125176, filed Oct. 18, 2018, and titled, “Trocars,” U.S. Patent Publication No. 2021/0127963, filed Nov. 21, 2019, and titled “Intraoperative Endoscope Cleaning System,” and U.S. Patent Publication No. 2021/0127964, filed Nov. 21, 2019, and titled “Intraoperative Endoscope Cleaning System,” the disclosure of each of which is hereby incorporated by reference in its entirety. Systems, devices, and methods described herein improve on such cleaning systems, e.g., by improving delivery of liquid and/or gas into a trocar, adding components for coupling liquid and/or gas lines, and improving venting of excess gases within a body lumen or cavity via a trocar.

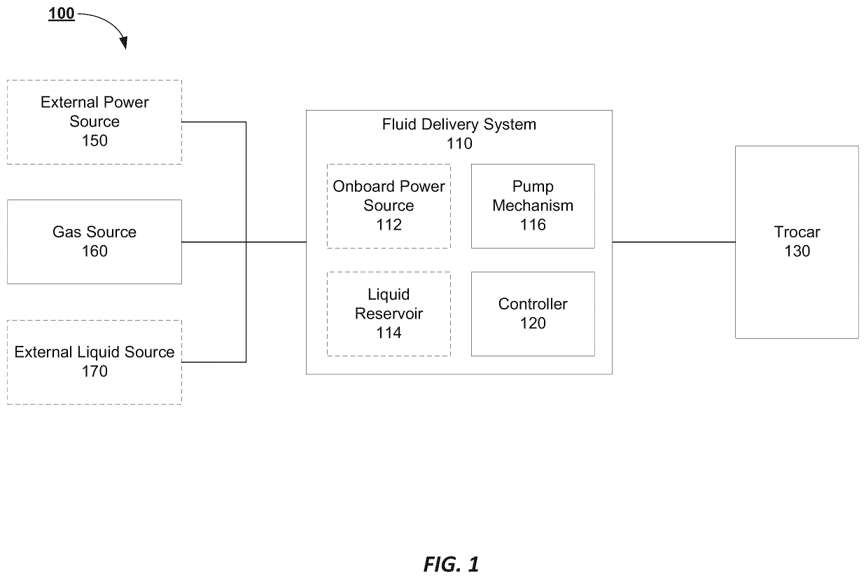

FIG. 1 is a block diagram of a system 100 for cleaning an endoscope or scope that can be disposed in a body lumen or cavity, according to an embodiment. As shown, the system 100 includes a fluid delivery system 110 fluidically coupled to a trocar 130 (or other elongate device) and a gas source 160. The fluid delivery system 110 can optionally include an onboard power source 112. Alternatively or additionally, the fluid delivery system 110 can optionally be coupled to an external power source 150. The fluid delivery system 110 includes a pump mechanism 116 and a controller 120. The fluid delivery system 110 can optionally a liquid reservoir 114. Alternatively or additionally, the fluid delivery system 110 can optionally be coupled to an external liquid source 170. Lines depicted in FIG. 1 connecting units can represent electrical, physical, and/or fluidic couplings.

The onboard power source 112 is an optional component integrated into the fluid delivery system 110. The onboard power source 112 powers the pump mechanism 116 and/or the controller 120. In some embodiments, the onboard power source can include a battery. In some embodiments, the onboard power source 112 can include a fuel cell. In some embodiments, the onboard power source can be integrated into the same structure as the liquid reservoir 114, the pump mechanism 116, and/or the controller 120. For example, the onboard power source 112, the liquid reservoir 114, and the pump mechanism 116 can be disposed together in a housing (or one or more housing sections that couple together to form a housing).

Optionally, an external power source 150 can be coupled to the fluid delivery system 110 to deliver power to one or more components of the fluid delivery system 110. In some embodiments, the external power source 150 can include a wall outlet. In some embodiments, the external power source 150 can include a battery or a battery pack physically separated from the fluid delivery system 110. In some embodiments, the external power source 150 can power the pump mechanism 116, the controller 116, and/or the onboard power source 112.

The liquid reservoir 114 is an optional component integrated into the fluid delivery system 110. The liquid reservoir 114 is configured to contain a liquid (e.g., wash liquid or solution), e.g., for cleaning an endoscope. In some embodiments, the washing fluid can include a saline solution, a buffered solution, a bio-compatible surfactant, and/or any other suitable wash solution, including those described in U.S. Patent Publication No. 2021/0127963. The liquid reservoir 114 can be configured to contain a volume of liquid that is sufficient for conducting at least about 5, at least about 10, at least about 50, at least about 100, at least about 500, at least about 1000, at least about 1500, or at least about 2000 wash sequences. For example, the liquid reservoir 114 can be filled with enough liquid for cleaning an endoscope throughout the duration of a surgical procedure. In some embodiments, the liquid reservoir 114 can be pre-filled with different volumes of liquid, e.g., depending on the estimated number of times that an endoscope positioned within a body lumen or cavity may need to be cleaned. Therefore, for longer procedures that may require a greater number of wash sequences, the liquid reservoir 114 may be filled with a greater volume of liquid. In some embodiments, the liquid reservoir 114 can have a volume of at least about 5 mL, at least about 10 mL, at least about 15 mL, at least about 20 mL, at least about 25 mL, at least about 30 mL, at least about 35 mL, at least about 40 mL, at least about 45 mL, at least about 50 mL, at least about 55 mL, at least about 60 mL, at least about 65 mL, at least about 70 mL, at least about 75 mL, at least about 80 mL, at least about 85 mL, at least about 90 mL, or at least about 95 mL. In some embodiments, the liquid reservoir 114 can have a volume of no more than about 100 mL, no more than about 95 mL, no more than about 90 mL, no more than about 85 mL, no more than about 80 mL, no more than about 75 mL, no more than about 70 mL, no more than about 65 mL, no more than about 60 mL, no more than about 55 mL, no more than about 50 mL, no more than about 45 mL, no more than about 40 mL, no more than about 35 mL, no more than about 30 mL, no more than about 25 mL, no more than about 20 mL, no more than about 15 mL, or no more than about 10 mL. Combinations of the above-referenced volumes of the liquid reservoir 114 are also possible (e.g., at least about 5 mL and no more than about 100 mL or at least about 20 mL and no more than about 40 mL), inclusive of all values and ranges therebetween. In some embodiments, the liquid reservoir 114 can have a volume of about 5 mL, about 10 mL, about 15 mL, about 20 mL, about 25 mL, about 30 mL, about 35 mL, about 40 mL, about 45 mL, about 50 mL, about 55 mL, about 60 mL, about 65 mL, about 70 mL, about 75 mL, about 80 mL, about 85 mL, about 90 mL, about 95 mL, or about 100 mL.

Alternatively or additionally, an external liquid source 170 can be used to supply liquid to the fluid delivery system 110. The external liquid source 170 be separate from the fluid delivery system 110 but coupled to the fluid delivery system 110, e.g., via a fluid line. In some embodiments, the external liquid source 170 can be a fluid bag or other type of fluid containing element. In some embodiments, the external liquid source 170 can be a water line or other fluid line within a building that can be coupled via a faucet or other connection to the fluid delivery system 110. In some embodiments, the external liquid source 170 can be used to fill (e.g., pre-fill or re-fill) the liquid reservoir 114. In some embodiments, the liquid delivered to the liquid reservoir can be the wash liquid or solution.

The pump mechanism 116 aids in delivering liquid (e.g., wash liquid or solution) to the trocar 130. In some embodiments, the pump mechanism 116 can include or form part of a centrifugal pump, peristaltic pump, lobe pump, rotary gear pump, horizontal split case pump, air operated pump, diaphragm pump, magnetically driven pump, a mechanically driven pump, an electrically driven pump, or any other suitable pump apparatus or combinations thereof. In a specific embodiment, the pump mechanism 116 can include a plunger, platform, shaft, or other suitable component that can be actuated (e.g., via a pump actuator 226) to compress a fluid line to deliver a liquid. For example, a pump mechanism 116 implemented as a plunger can be actuated to compress a flexible housing or tubing that contains a liquid. The compression of the flexible housing or tubing can cause the liquid within the flexible housing or tubing to be driven toward the trocar 130, e.g., to fill the lines for a wash or cleaning sequence.

The controller 120 controls operation of the pump mechanism 116. In some embodiments, the controller 120 can be in communication with or include a processor and/or a user interface. Operation of the pump mechanism 116 can be automatic or user-controlled. In some embodiments, the user via the user interface can set parameters for when to activate the pump mechanism 116, e.g., to supply additional liquid for cleaning an endoscope. In some embodiments, the controller 120 can activate the pump mechanism 116 after each wash sequence to fill the liquid lines for a subsequent wash sequence. In some embodiments, the controller 120 can activate the pump mechanism 116 to fill the liquid lines in response to an indication that an endoscope has been positioned for cleaning (e.g., based on signals received by the controller 120 from one or more sensors). In some embodiments, the controller 120 can activate the pump mechanism 116 to fill the liquid lines in response to a detection of a drop in pressure or volume in the liquid lines (e.g., based on signals received by the controller 120 from one or more sensors).

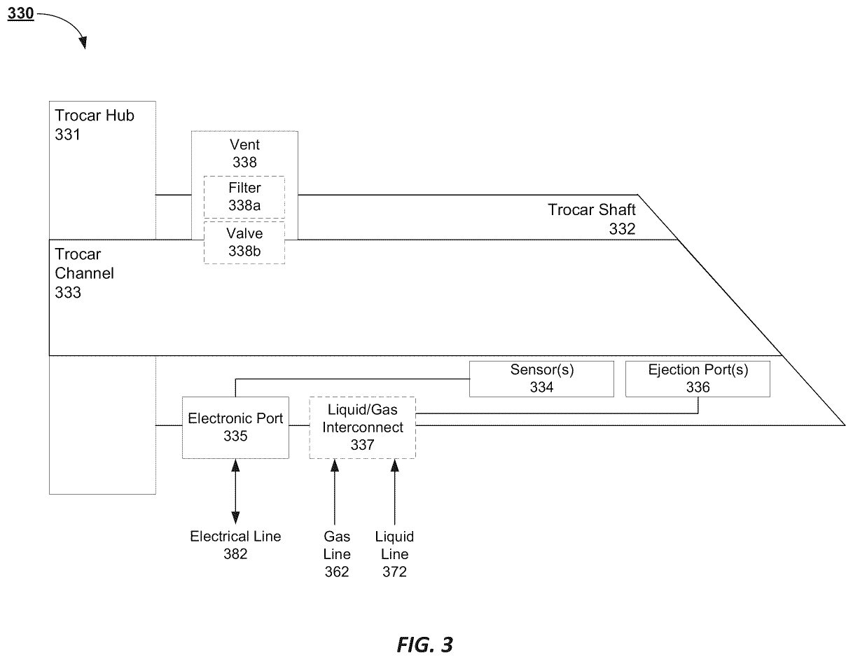

The trocar 130 can be a surgical instrument that can be placed within a patient to provide access into a body lumen or cavity of the patient. In use, the trocar 130 is placed in the body lumen or cavity, e.g., with or without an obturator. The body lumen or cavity can include a thoracic cavity or an abdominal cavity. The trocar 130 includes a shaft or elongate structure that defines a channel or lumen within which an endoscope for viewing of the inside of the body lumen or cavity can be positioned. The trocar 130 includes one or more channels or lines in fluidic communication with the fluid delivery system 110, e.g., for receiving gas and/or liquid from the fluid delivery system 110. In some embodiments, the channels or lines can be primed after each deployment of wash solution (e.g., after each wash sequence). In some embodiments, the trocar 130 can be maintained in place by an anchor (not shown) placed on the outside of the patient's body. Further details of an example trocar are provided with reference to FIG. 3 below.

The gas source 160 is used to pressurize the liquid and deliver the liquid via the fluid delivery system 110. In some embodiments, the gas source 160 can include a container (e.g., a tank) that houses a volume of pressurized gas. In some embodiments, the gas source 160 can deliver gas at a pressure of between about 20 psi and about 50 psi, including all values and sub-ranges therebetween. In some embodiments, the gas delivered by the gas source 160 can include air, CO₂, nitrogen, argon, or any other inert gas or combinations thereof. The selected gas can be a gas that is commonly used in medical procedures and is safe for delivery into a body lumen or cavity.

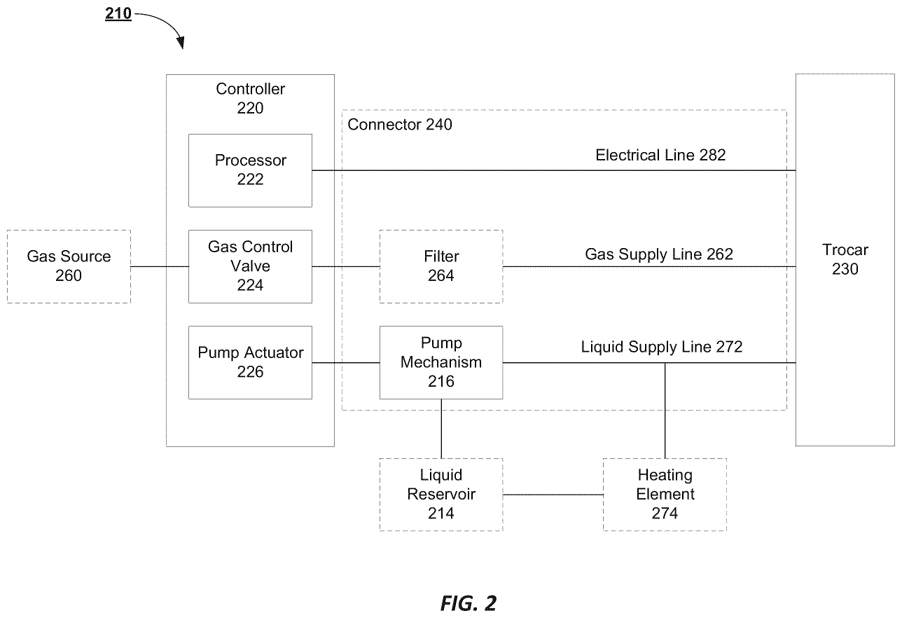

FIG. 2 provides a more detailed view of the liquid and gas connections of a fluid delivery system 210 of a cleaning system for cleaning an endoscope disposed in a body lumen or cavity, according to an embodiment. The fluid delivery system 210 can be structurally and/or functionally similar to other fluid delivery systems described herein, including, for example, fluid delivery system 110. As shown, the fluid delivery system 210 includes a controller 220. The fluid delivery system 219 is fluidically coupled to the trocar 230 and an optional gas source 260. The controller 220 includes a processor 222, a gas control valve 224, and a pump actuator 226. A liquid reservoir 214 can be fluidically coupled to a liquid supply line 272, with a pump mechanism 216 disposed along the coupling or line to control delivery of the liquid. In some embodiments, an optional heating element 274 can be coupled to the liquid reservoir 214 and/or the liquid supply line 272, e.g., for heating the liquid. A gas source 260 can be fluidically coupled to a gas supply line 262, with a gas control valve 224 disposed along the coupling or line to control delivery of the gas.

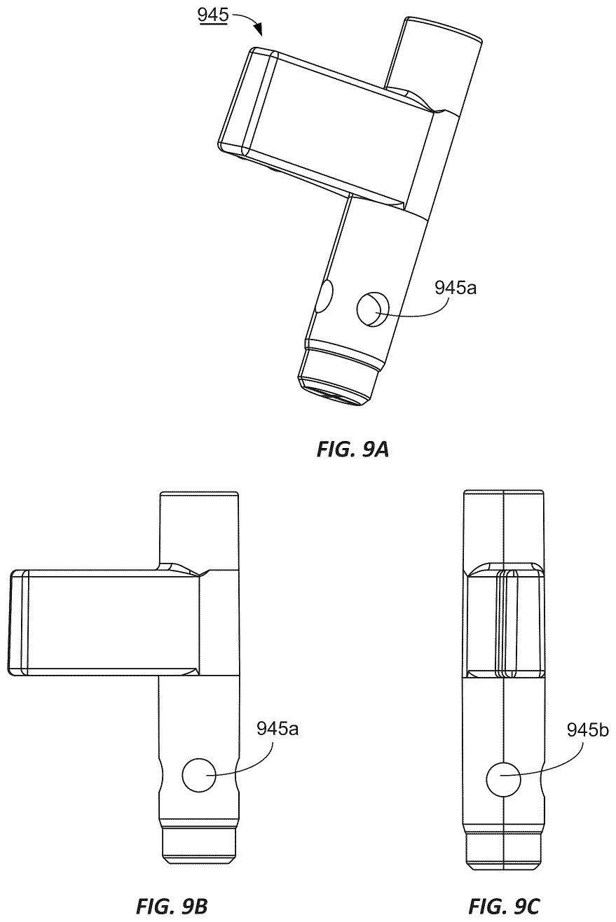

In some embodiments, an optional connector or cable 240 can house the gas supply line 262, the liquid supply line 272, and the electrical line 282. In some embodiments, the connector 240 can include an outer cylindrical or tubular housing (e.g., a cable housing) that defines a lumen for containing the gas supply line 262, the liquid supply line 272, and the electrical line 282. In some embodiments, the connector 240 can be composed of an insulative material, a rubber, a plastic, a polymer, or any combination thereof. The connector 240 can include a proximal connection or controller connection and a distal connection or trocar connection that each include connecting elements for coupling to the controller 220 and the trocar 230, respectively. Further details of a connector are described with reference to FIGS. 9C and 9D.

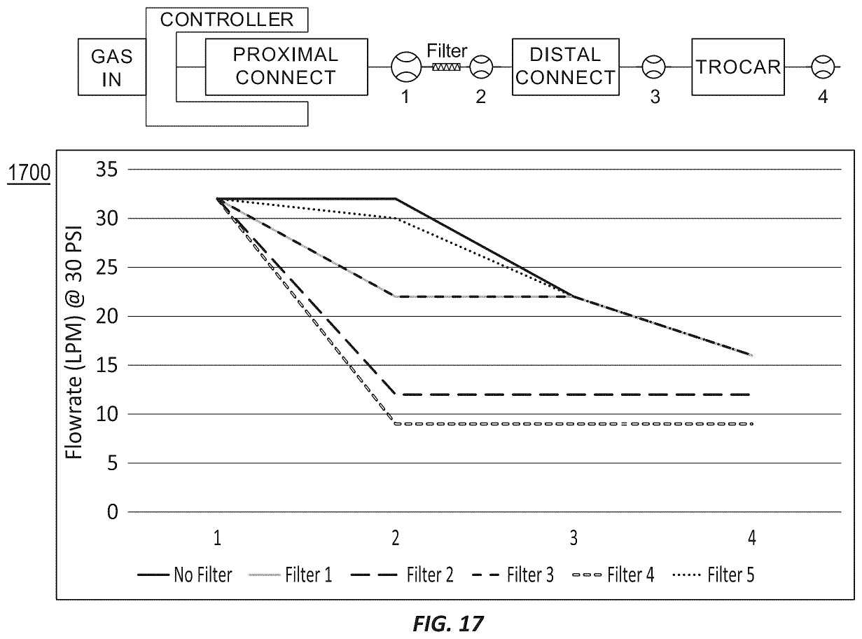

In some embodiments, an optional filter 264 can be integrated into the gas supply line 262. The filter 264 can be configured to filter the gas prior to delivery into a body lumen or cavity, e.g., to prevent contaminants, viruses, bacteria, etc. from entering the body lumen or cavity. In some embodiments, the filter 264 can be selected to provide filtering while also not disrupting the pressure behavior of the cleaning system. In particular, when pressure is switch on in the cleaning system (e.g., via opening of valve 224), the pressure may cause a pressure spike that can beneficially produce a desirable spray of liquid within a channel of the trocar 230. The presence of a filter 264, however, may affect the behavior of the pressure of the pressurized gas. FIG. 17 shows the impact that different filters have on flow rates, according to embodiments. Five different types of filters were tested, including Qosina 11679 (Filter 1), Pall Acrylic 6664197 (Filter 2), Qosina 28204 (Filter 3), Pall Posidyne GELELD96LL (Filter 4), Sartorius 17805—Commercial Embodiment (Filter 5). As shown, the filters are placed between a proximal connection and a distal connection of a connector. The flow rates at several locations along the gas line extending from a gas inlet (GAS IN) to a trocar are then measured. Specifically, the flow rates at each of points 1, 2, 3, and 4 are measured. The resulting data showing the change in flow rates along the gas lines are depicted in plot 1100. As shown, Filter 5 resulted in less disruption in flow rate along the gas line and therefore performed better than the other filters for the purposes of being used in the cleaning systems described herein. In some embodiments, the filter can be configured to filter the gas without changing a flow rate of the gas by more than about 10%, including any values or sub-ranges less than that. In some embodiments, a filter with a pore size of between about 0.1 and about 0.2 μm, including all values and sub-ranges therebetween, may be used. In some embodiments, the filter may be made from a polymer, ceramic, or glass material, including, for example, polycarbonate, polytetrafluoroethylene, acrylic, micro-glass, ceramic fiber, polypropylene, polyethersulfone, or combinations thereof.

While the filter 264 is described as being disposed in the gas supply line, it can be appreciated that one or more other filters can be used with the systems and devices described herein, including, for example, a filter disposed in the liquid supply line for filtering the liquid that is delivered to the trocar.

The processor 222 can be coupled to an electrical line 282, e.g., for sending and/or receiving data from electrical elements disposed in the trocar. For example, the processor 222 via the electrical line 282 can be configured to receive data from one or more sensors disposed along a channel of the trocar, e.g., to detect when an endoscope is being retracted within the trocar for initiating a wash sequence. Optionally, the gas supply line 262, the liquid supply line 272, and the electrical line 282 can be contained within a connector 240. The connector 240 can be a cable that houses each of the lines. In some embodiments, the liquid reservoir 214, the pump mechanism 216, the controller 220, and the gas source 260 can be the same or substantially similar to the liquid reservoir 114, the pump mechanism 116, the controller 120, and the gas source 160, as described above with reference to FIG. 1. Thus, certain aspects of the liquid reservoir 214, the pump mechanism 216, the controller 220, and the gas source 260 are not described in greater detail herein.

The processor 222 is electrically connected to the trocar 230 via the electrical line 282. In some embodiments, the processor 222 can communicate with sensor(s) (not shown) in the trocar 230. The processor 222 can receive information from the sensor(s) and, based on that information, control the delivery of the gas and/or liquid, e.g., for initiating a wash sequence. For example, one or more sensors disposed in the trocar 230 can detect a position of the distal end of an endoscope positioned within the trocar. When the sensor(s) detect that the endoscope is retracted and/or positioned for cleaning, the sensor(s) can send that data to the processor 222, which can activate the delivery of gas and/or liquid to clean the distal end of the endoscope. In some embodiments, the electrical line 282 can include conductive wiring. In some embodiments, the conductive wiring can be composed of copper, silver, brass, gold, titanium, stainless steel, carbon steel, or any combination thereof. In some embodiments, the electrical line 282 can be housed within the connector 240 and insulated from external elements.

The gas source 260 and the gas control valve 224 are fluidically coupled to the trocar via the gas supply line 262. The gas control valve 224 controls delivery of gas into the trocar 230. The gas control valve 224 can be controlled by the controller 220 (specifically, processor 222) to deliver gas at desired times, e.g., when an endoscope is positioned for cleaning. In some embodiments, the gas supply line 262 can include flexible tubing. In some embodiments, the tubing can be composed of a polymer, polyvinylchloride (PVC), polyurethane, Tygon®, acrylic, or any other suitable material. In some embodiments, the gas supply line 262 can include a filter (e.g., filter 264), e.g., for filtering the gas prior to delivery to the trocar.

The pump actuator 226 can actuate the pump mechanism 216 to deliver liquid to the trocar 230 via the liquid supply line 272. The pump actuator 226 can include an electrical motor and/or other drive mechanisms for actuating the pump mechanism 216. In some embodiments, the pump mechanism 216 can be a plunger, shaft, or other suitable structure that can be actuated to compress flexible tubing containing the liquid, e.g., to pump the liquid. In some embodiments, the pump actuator 226 can be powered by a battery, a wall outlet, or any other suitable power mechanism. In some embodiments, the liquid supply line 272 can include flexible tubing. In some embodiments, the tubing can be composed of a polymer, PVC, polyurethane, Tygon®, acrylic, or any other suitable material. Optionally, the heating element 274 can provide heat to the liquid reservoir 214 and/or the liquid supply line 272. In some embodiments, the heating element 274 can be configured to heat the liquid to body temperature such that delivery of the liquid into the trocar does not cause fogging of the distal end of an endoscope, e.g., due to a temperature difference between the liquid and the gases within the body lumen or cavity. In some embodiments, the heating element 274 can provide heat to facilitate fluid flow of liquid through the liquid supply line 272 (e.g., by lowering the viscosity of the liquid).

FIG. 3 is a diagram of a trocar 330, according to an embodiment. As shown, the trocar 330 includes a trocar hub 331 and a trocar shaft 332. A trocar channel 333 extends through the trocar shaft 332. One or more sensors 334, an electronic port 335, one or more ejection ports 336, and/or a liquid/gas interconnect or interconnector 337 are integrated into or disposed in the trocar shaft 332. A vent 338, optionally including a filter 338a and a valve 338b, can also be integrated into or disposed in the trocar shaft 332. An electrical line 382 can be coupled to the electronic port 335, while a gas line 362 and a liquid line 372 can be coupled to the liquid/gas interconnect 337. In some embodiments, the gas line 362, the liquid line 372, and the electrical line 382 can be the same or substantially similar to the gas supply line 262, the liquid supply line 272, and the electrical line 282, as described above with reference to FIG. 2. Thus, certain aspects of the gas line 362, the liquid line 372, and the electrical line 382 are not described in greater detail herein.

The trocar hub 331 is an enlarged portion of the trocar 330 for housing one or more components of the trocar 330. The trocar hub 331 provides a handle for placement of the trocar 330. The trocar shaft 332 is an elongated portion of the trocar 330 and is connected to the trocar hub 331. In use, the trocar hub 331 can be positioned outside of a patient's body while the trocar shaft 332 (or a substantial majority of the trocar shaft 332) is positioned within the patient's body.

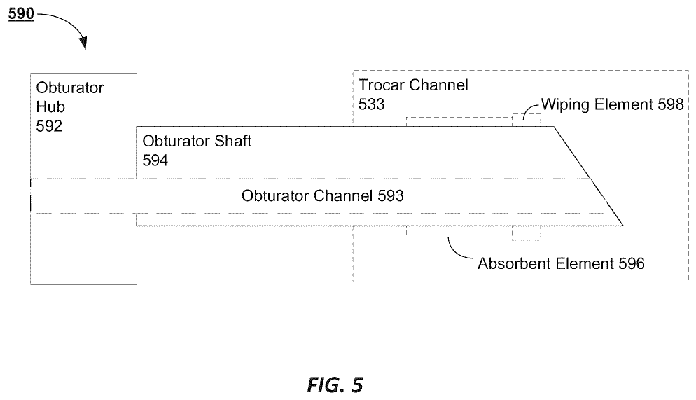

The trocar hub 331 and the trocar shaft 332 can collectively define a trocar channel 333 for receiving an instrument, e.g., an endoscope, an obturator, etc. In some embodiments, the trocar channel 333 can have a diameter of between about 1 mm (3 French) and about 10 mm (30 French), including all sub-ranges and values therebetween. For example, the trocar channel 333 can have a diameter of about 10 French or slightly larger than 10 French such that the trocar channel 333 is configured to receive an instrument (e.g., endoscope) having up to a 10 French diameter. In use, the trocar 330 can be positioned within a patient such that the channel 333 extends into a body lumen or cavity. The channel can therefore provide access to a body lumen or cavity, e.g., for positioning one or more instruments within the body lumen or cavity. The trocar 330 can be positioned through an incision in the patient's body. In some embodiments, an obturator (e.g., obturator 590) can be positioned within the trocar channel 333 while the trocar is being positioned within the body and then removed after the distal end of the trocar has been positioned within the body lumen or cavity. Other instruments (e.g., endoscopes) can then be positioned within the trocar channel 333 after the obturator has been removed. Further details of an obturator are described with reference to FIG. 5.

The trocar 330 can form a part of a cleaning system for an endoscope, e.g., such as the system described above with reference to FIG. 1. As such, the trocar 330 can include components that can facilitate a cleaning or wash sequence associated with an endoscope. In particular, the trocar 330 an include one or more ports (e.g., electronic port 335, ejection port(s) 336, liquid/gas port(s)) and/or one or more sensor(s) 334.

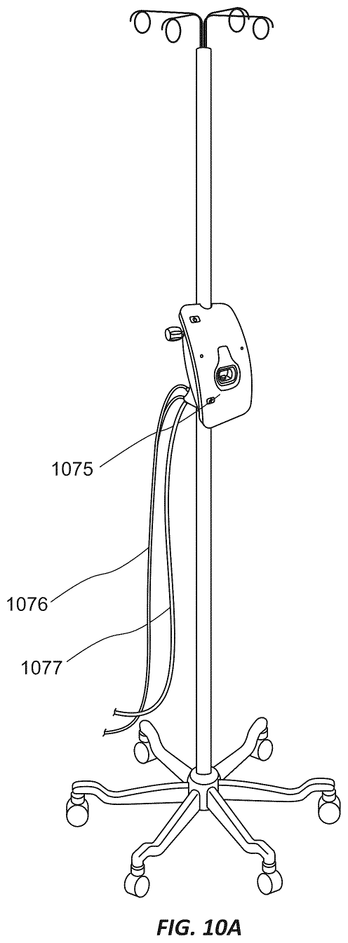

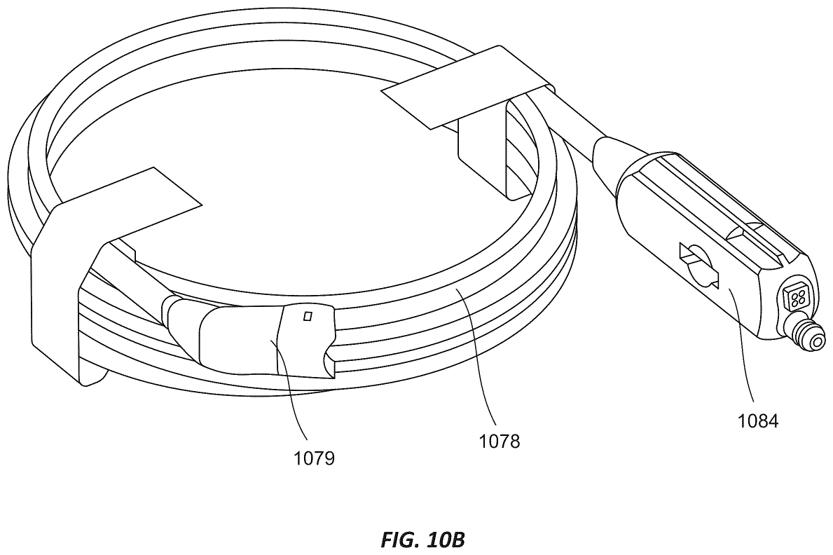

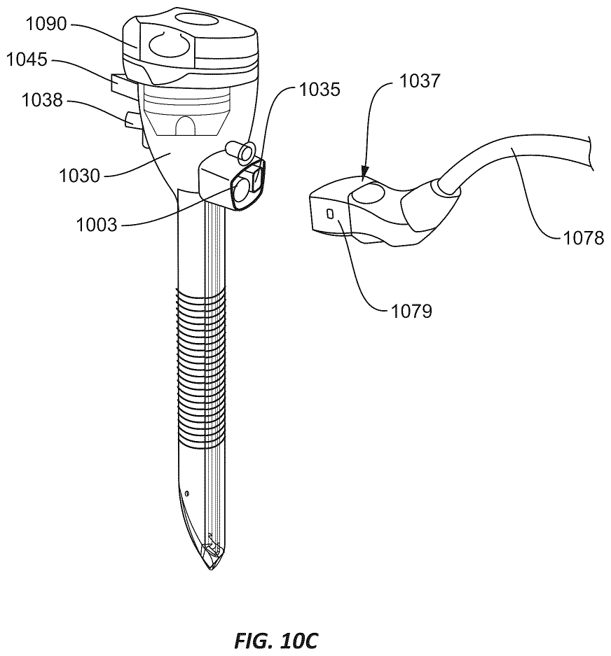

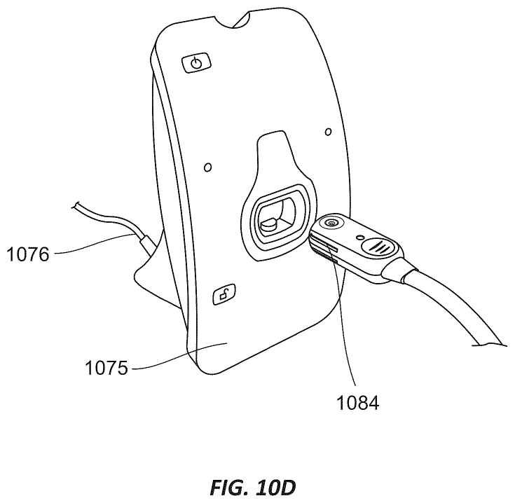

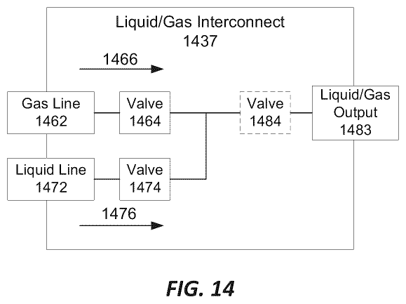

In some embodiments, the trocar 330 can optionally include a liquid/gas interconnect 337. The liquid/gas interconnect 337 can be configured to combine a liquid stream and a gas stream into one output stream, as further described with reference to FIG. 14. The liquid/gas interconnect 337 receives a feed from the gas line 362 and the liquid line 372. The liquid/gas interconnect 337 can include a collection of valves and tubes for controlling the delivery of fluid (e.g., gas and/or liquid). In some embodiments, the liquid/gas interconnect 337 can be integrated into or disposed in a connector that coupled to the trocar 330 (e.g., a trocar connection or distal connection of connector 240) instead of being integrated into or disposed in the trocar 330. In such embodiments, the output stream from the liquid/gas interconnect 337 can be coupled to a liquid/gas port integrated into or disposed in the trocar shaft 332. Further details of such a configuration are described with reference to FIGS. 10A-10D.

The sensor(s) 334 can detect whether a device (e.g., an obturator, a scope) is in the trocar channel 333 and/or a position and/or orientation of the device within the trocar channel 333. The sensor(s) 334 can trigger liquid and/or gas deployment via the ejection port(s) 336 upon detecting that the device is in a position and/or orientation for cleaning. For example, when a device is retracted into the trocar channel 333 such that at least one sensor 334 detects the device, the sensor(s) 334 can trigger liquid and/or gas deployment. The sensor(s) 334 can be coupled to a controller (e.g., controller 220) via electronic port 335 and electrical line 382. As such, the sensor(s) 334 can send signals to the controller for detecting a position and/or orientation of the device. In response to detecting that the device is in a position and/or orientation for cleaning, the controller can trigger delivery of the liquid and/or gas via one or more ejection port(s) 336 into the trocar channel 333. In some embodiments, the trocar 330 can include 1, 2, 3, 4, 5, 6, 7, 9, 10, or at least about 10 sensors 334. For example, in some embodiments, the trocar 330 can include a single sensor 334 that can be configured to detect when an instrument (e.g., an endoscope) is close to the sensor (e.g., based on light detected by the sensor being above a predetermined threshold), and in response to detecting the light, the liquid and/or gas delivery can be triggered (e.g., via a controller). In some embodiments, the trocar 330 can include a first sensor that detects when an instrument (e.g., endoscope) is first inserted into the trocar channel 333 and a second sensor that detects when the instrument, having been previously inserted into the trocar channel 333, is being retracted for cleaning. In such embodiments, the first sensor, upon detecting that the instrument is being inserted into the trocar channel 333, can send a signal to a controller to not initiate a wash sequence as the instrument passes by the second sensor. Then with subsequent detection of the instrument by the second sensor (e.g., in response to a retraction of the instrument), the second sensor can send a signal to the controller to initiate the wash sequence. The sensor(s) 331 can include one or more light sensors, photoelectric sensors, pressure sensors, infrared sensors, force sensors, position sensors, piezoelectric sensors, mechanical sensors, etc.

The electronic port 335 can couple the sensor(s) 334 to the electrical line 382 and other electronic components of a cleaning system (e.g., controller 220). In some embodiments, the electronic port 335 is configured to provide power to the sensor(s) 334. In some embodiments, the electronic port 335 is configured to send to and/or receive data from the sensor(s) 334.

The ejection port(s) 336 eject a gas and/or liquid (e.g., a wash solution) into the trocar channel 333, e.g., to wash a device such as, for example, an endoscope positioned in the trocar channel. In some embodiments, the ejection port(s) 336 can be angled retrograde or back towards a proximal end of the trocar 330 such that the ejection port(s) 336 eject the gas and/or liquid in a proximal direction, e.g., toward a distal end of an instrument. In some embodiments, the trocar 330 can include a single ejection port that is configured to generate a spray, e.g., for cleaning a distal end of an endoscope. In some embodiments, the trocar 330 can include a plurality of ejection ports for generating sprays. In some embodiments, the plurality of ejection portions can be set at different angles and/or orientations to cover a larger region within the trocar channel 333.

In use, a high-pressure source of gas can be used to deliver a set volume of liquid (e.g., wash solution) into the trocar channel 333. The high-pressure source of gas and the liquid can be coupled via the gas line 362 and the liquid line 372, respectively, to the liquid/gas interconnect 337. The liquid/gas interconnect 337 can combine the high-pressure gas with the liquid, and with each wash sequence, allow the high-pressure gas to draw and eject a set volume of liquid into the trocar channel 333. In some embodiments, the high-pressure gas can be delivered at pressures of at least about 20 psi to at least about 50 psi, including all sub-ranges and values therebetween. For example, in an embodiment, the high-pressure gas can be delivered at a pressure of at least about 30 psi, at least about 35 psi, or at least about 40 psi. Each wash sequence can last about 100 to about 500 ms, including all sub-ranges and values therebetween. For example, in an embodiment, the wash sequence can be at least about 100 ms to about 300 ms, including 200 ms.

The liquid being delivered by the ejection port(s) 336 can include water, a saline solution, a buffered solution, or a bio-compatible surfactant. For example, the liquid or wash solution can include a mixture of water and a surfactant. The mixture can include at least about 10% surfactant, about 15% surfactant, about 20% surfactant, about 25% surfactant, about 30% surfactant, about 35% surfactant, about 40% surfactant, or higher amounts of surfactant to water. In use with cleaning an endoscope, the distal end of the endoscope can be coated with a surfactant solution before being inserted into the trocar channel 333. The wash solution with a percentage of surfactant can then be used to wash the distal end of the endoscope, e.g., in one or more wash sequences when the endoscope is retracted. The presence of the surfactant in the wash solution can build a hydrophobic layer on the distal end of the endoscope, which can reduce fogging, water build-up, and other types of build-up on the distal end of the endoscope.

The vent 338 is fluidically coupled to the trocar channel 333. The vent 338 can be configured to allow for release for gases built up during surgery. In some embodiments, the vent 338 can be a passive vent, e.g., an opening that can allow gases to exit the body lumen or cavity via the trocar channel 333 and vent 338. Alternatively, the vent 338 can be coupled to a vacuum source or other active component that can be used to regulate pressure within the body lumen or cavity. As shown, the vent 338 can optionally include a filter 338a and a valve 338b. The filter 338a can capture filter the gases exiting the body lumen or cavity and reduce smell or other compounds being carried by the existing gases. The filter 338a can be configured to vent gases and/or liquids, including, for example, water vapors, as well as gases and smoke created during tissue cutting and/or coagulation using electrosurgical devices (e.g., radiofrequency (RF) and/or ultrasonic cutting tools). The valve 338b can be opened or closed to permit or block gas flow through the vent 338. In some embodiments, the valve 338b can be controlled manually. For example, a physician can open or close the valve 338b via a switch, button, etc. to allow for venting. In some embodiments, the valve 338b can be configured to automatically open, e.g., when the pressure within the body lumen or cavity is above a predefined pressure. The valve 338b can also be configured to prevent backflow, e.g., flow of air or other gases within an external environment around the trocar into the trocar lumen 333 and/or body lumen or cavity.

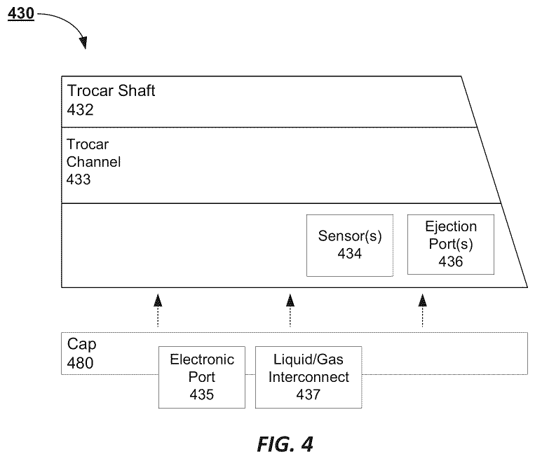

FIG. 4 depicts an example trocar 430, where the trocar 430 includes a main shaft 432 and a cap 480, according to an embodiment. As shown, the trocar shaft 432 can include a trocar channel 433 extending therethrough, a sensor 434, and an ejection port 436. The cap 480 can be formed separately from the trocar shaft 432 but be coupled to the trocar shaft 432. The cap 480 can include an electronic port 435 and a liquid/gas interconnect 437. In some embodiments, the trocar 430, the trocar shaft 432, the trocar channel 433, the sensor 434, and the ejection port 436 can be the same or substantially similar to the trocar 330, the trocar shaft 332, the trocar channel 333, the sensor(s) 334, and the ejection port 336, as described above with reference to FIG. 3. Thus, certain aspects of the trocar 430, the trocar shaft 432, the trocar channel 433, the sensor 434, and the ejection port 436 are not described in greater detail herein.

As noted, the cap 480 can be coupleable to the trocar shaft 432. In some embodiments, when the cap 480 is coupled to the trocar shaft 432, the cap 480 and the trocar shaft 432 can collectively form a passage for directing liquid and/or gas from the liquid/gas interconnect 437 to the ejection port(s) 436 and/or a passage for housing an electrical connection between the electronic port 435 and the sensor(s) 434. As such, each of the trocar shaft 432 and the cap 480 can include grooves or ridges that define such passages. Alternatively, in some embodiments, the cap 480 can define one or more portions of a passage for directing liquid and/or gas from the liquid/gas interconnect 437 to the ejection port(s) 436 and/or a passage for housing an electrical connection between the electronic port 435 and the sensor(s) 434, while the trocar shaft 432 can define other portions of such passages. In such cases, when the cap 480 and the trocar shaft 432 are coupled together, the respective portions of the passages defined by the cap 480 and the trocar shaft 432 can join together. Still alternatively, in some embodiments, the cap alone can define (or substantially define) a passage for directing liquid and/or gas from the liquid/gas interconnect 437 to the ejection port(s) 436 and/or a passage for housing an electrical connection between the electronic port 435 and the sensor(s) 434. Still alternatively, in some embodiments, the trocar shaft 432 can define a passage for directing liquid and/or gas from the liquid/gas interconnect 437 to the ejection port(s) 436 and/or a passage for housing an electrical connection between the electronic port 435 and the sensor(s) 434.

In some embodiments, the cap 480 can fit into the trocar shaft 432 via an interlocking mechanism. For example, the cap 480 can including coupling elements that snap into place on the trocar shaft 432. In some embodiments, the cap 480 can be coupled to the trocar shaft 432 via a mechanical, magnetic, adhesive, etc. manner. In some embodiments, the cap 480 can fit into grooves on the trocar shaft 432. In some embodiments, the cap 480 can be formed separately from the shaft 432 but be permanently coupled to the shaft 432 to form a unitary piece. In some embodiments, the cap 480 can be coupled to and decoupled from the shaft 432. In some embodiments, the electronic port 435 and the liquid/gas interconnect 437 can be integrated into the cap 480. The liquid/gas interconnect 437 can be fluidically connected to ejection port(s) 436 in the trocar shaft 432 when the cap 480 and the trocar shaft 432 and coupled together. The electronic port 435 can include a mating surface for an electrical plug or other connection. In some embodiments, the electronic port 435 and the sensor(s) 434 can be communicatively coupled to one another via a flat or flexible circuit board. The flexible circuit board can be disposed in a passage defined by one or both of the cap 480 or trocar shaft 432, as described above.

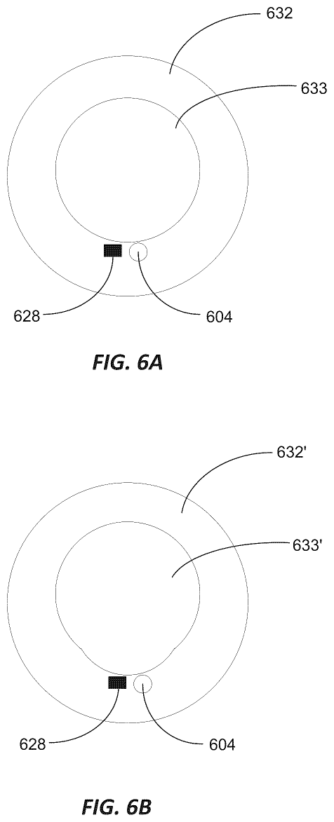

FIGS. 6A-6B show cross-sectional views of a trocar shaft 632, according to an embodiment. FIG. 6A shows a trocar shaft 632 with a trocar channel 633 having a circular cross section, while FIGS. 6B shows a trocar shaft 632′ with a trocar channel 633′ with a non-circular cross-section. The trocar channel 633′ can be referred to as having an asymmetrical cross-section. In particular, the asymmetrical cross-section can be formed of a plurality of side walls or portions with a side wall or portion that extends further out radially than one or more other side walls or portions. An electrical line (e.g., a printed circuit board (PCB)) 628 and a liquid and/or gas lumen 604 can be disposed adjacent to the trocar channels 633, 633′. The asymmetrical shape of the trocar channel 633, 633′ can create a space or provide a gap between the wall of the trocar channel 633, 633′ and the wall of the camera when a circular endoscope is placed inside the trocar 630. This design can prevent the trocar channel 633, 633′ from being blocked by a body of the camera, such that a space is always present between the body of the camera and the walls of the trocar channel 633, 633′, such that the outer surface of the camera is not able to rest on the wall of the trocar channel 633, 633′. This can help prevent fluid buildup inside the trocar 630.

In some embodiments, the electrical line 628 can supply power to one or more electrical components (e.g., sensors) disposed within the trocar shafts 632, 632′. In some embodiments, the electrical line 628 can receive data from and/or send data to one or more electrical components (e.g., sensors) disposed within the trocar shafts 632, 632′. The liquid and/or gas lumen 604 can deliver a liquid (e.g., wash solution) or a gas to an ejection port (not shown) at a distal end of the trocar 630.

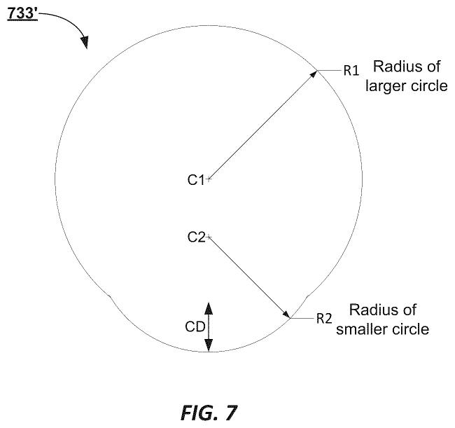

The trocar channel 633′ can be asymmetrical such that additional clearance can be provided between an exit of an ejection port and the surface of an endoscope. FIG. 7 provides a more detailed breakdown of the cross section of an asymmetrical trocar channel 733′, according to an embodiment. As shown, the trocar channel 733′ results from the combination of a first circular cross-section having a first radius R1 and a first center C1 and a second circular cross-section having a second radius R2 and a second center C2. In some embodiments, the first circular cross-section can be the cross-section that receives a portion of an endoscope. In other words, an endoscope that is positioned within the trocar channel 633′ can be configured to sit within the first circular cross-section with radius R1. The first radius R1 can be greater than the second radius R2.

In some embodiments, the first radius R1 can be about 1.1 to about 1.5 times the second radius R2, including all values and sub-ranges therebetween. In some embodiments, the first radius R1 can be between about 0.5 mm to about 10 mm, including all values and sub-ranges therebetween. In some embodiments, the first cross-section can have a radius R1 that allows it to receive endoscopes of up to about 10 French (3.33 mm) or up to about 30 French (10 mm), including all values therebetween. For example, the first cross-section can have a diameter (i.e., two times the radius R1) that is slightly larger than the largest sized endoscope that the trocar channel 633′ is designed to receive. In some embodiments, the second radius R2 can be between about 0.1 mm to about 5 mm, including all values and sub-ranges therebetween.

In some embodiments, the second center C2 can be offset from the first center C1 by at least about 0.5 mm, at least about 1 mm, at least about 2 mm, at least about 3 mm, at least about 4 mm, or at least about 5 mm. In some embodiments, the second center C2 can be offset from the first center C1 by between about 0.5 mm and about 10 mm, including all values and sub-ranges therebetween. In some embodiments, the second center C2 can be offset from the first center C1 by a value of less than a radius R1 of the first cross-section. In other words, the first and second circular cross-sections can be designed to overlap, thereby forming the combined asymmetrical cross-section.

As shown, the second circle extends beyond the outer circumference of the first circle by a clearance distance CD. In some embodiments, the clearance distance CD can be at least about 0.1, at least about 0.5, at least about 1 mm, at least about 2 mm, at least about 3 mm, at least about 4 mm, at least about 5 mm, at least about 6 mm, at least about 7 mm, at least about 8 mm, at least about 9 mm, or at least about 10 mm. In some embodiments, the clearance distance CD can be no more than about 10 mm, no more than about 9 mm, no more than about 8 mm, no more than about 7 mm, no more than about 6 mm, no more than about 5 mm, no more than about 4 mm, no more than about 3 mm, no more than about 2 mm, or no more than about 1 mm. Combinations of the above-referenced values of the clearance distance CD are also possible (e.g., at least about 1 mm and no more than about 10 mm or at least about 1 mm and no more than about 2 mm), inclusive of all values and ranges therebetween.

The clearance distance CD can represent the distance between an exit of an ejection port (e.g., ejection port 336, 446) and an endoscope positioned within the trocar channel 633′. In use, an endoscope can be positioned within the first circular cross-section with the radius R1, and an ejection port can be disposed along the second cross-section with the radius R2. In some embodiments, the ejection port can be disposed in the second cross-section at the furthest point (or near furthest point) away from the first cross-section. For example, as shown in FIG. 6B, a liquid and/or gas channel 604 that is coupled to an ejection port can be disposed near a point in the cross-section that is furthest from the first cross-section. In some embodiments, the ejection port can be disposed at other points along the perimeter of the section cross-section. When a volume of liquid and/or gas is ejected via the ejection port, that volume can spread out or diffuse within the clearance distance CD before contacting the endoscope. As such, a trocar channel with an asymmetrical cross-section can be configured to allow for greater distribution of liquid and/or gas, which can enable greater contact between a liquid spray and the distal end of an endoscope. Additionally, the trocar channel with the asymmetrical cross-section can prevent closure or blocking of the ejection port by an endoscope (e.g., in the case of a tight fit between the endoscope and the walls of the trocar channel). The trocar channel with the asymmetrical cross-section can also prevent or reduce fluid buildup inside the trocar.

FIG. 5 is a diagram of an obturator 590, according to an embodiment. As shown, the obturator 590 includes an obturator hub 592, an obturator shaft 594, an optional obturator channel 593 extending the length of the obturator 590, an absorbent element 596, and a wiping element 598. As shown, the obturator 590 can be inserted into the trocar channel 533. In some embodiments, the trocar channel 533 can be the same or substantially similar to the trocar channel 333, as described above with reference to FIG. 3. Thus, certain aspects of the trocar channel 533 are not described in greater detail herein.

The obturator shaft 594 has sharp edges to facilitate initial placement of a trocar into the body lumen or cavity. For example, the distal end of the obturator shaft 594 can form a sharp, penetrating tip with a distal end of a trocar, and the penetrating tip can be used to cut through tissue while the trocar and obturator 590 are inserted through the tissue into the body lumen or cavity. In some embodiments, an imaging device such as an endoscope can be placed in the obturator channel 593 during placement of the obturator 590 in the body lumen or cavity. In such embodiments, the imaging device can be positioned within the obturator channel 593 and used to capture image data of the patient anatomy as the obturator 590 is inserted into the body lumen or cavity. In some embodiments, the obturator 590 can be composed of a polymer, polyethylene, polypropylene, PVC, polycarbonate, polystyrene, or any other suitable material.

The obturator hub 592 can optionally include a wiping element 598 and an absorbent element 596. In some embodiments, the wiping element 598 and/or the absorbent element 596 can be configured to clean an endoscope, e.g., when the endoscope is removed from a patient. For example, the wiping clement 594 and/or absorbent element 596 can be used to wipe the end of an endoscope, e.g., to remove debris or other deposits on the end of the endoscope. In some embodiments, the wiping element 598 and/or the absorbent element 596 can be configured to clean the trocar channel 533 and/or other parts of the trocar 530. For example, the wiping element 598 can contact a wall of the trocar channel 533 to remove moisture and debris from the wall. The wiping element 598 can also be configured to clean seal in the trocar 530. In some embodiments, the wiping element 598 can be implemented as an annular wiper blade that can encircle a perimeter of the obturator shaft 594. For example, the wiping element 598 can include a squeegee, a rubber stopper, an O-ring, or any other suitable device. When the obturator hub 592 is disposed within the trocar channel 533, the wiping element 598 can be configured to create a seal with the inner surface of the trocar channel 533. In some embodiments, the wiping element 594 can be directionally orientated such that insertion of the obturator 590 into the trocar channel 533 does not cause wiping but proximal retraction of the obturator 590 from the trocar channel 533 causes wiping or scraping of the sides of the trocar channel 533. In some embodiments, the wiping element 594 can be formed of a flexible material, while in other embodiments, the wiping element 594 can include portions that are formed of rigid material and/or portions that are formed of flexible material.

The absorbent element 596 can be configured to absorb debris and/or moisture from the interior sides of the trocar channel 533 and/or other parts of the trocar 530 (e.g., seals). The debris and/or moisture absorbed can include debris and/or moisture dislodged or captured by the wiping element 598. In some embodiments, the absorbent element 596 can include cloth, cotton, sponge, calcium chloride, silica gel, activated carbon, sodium polyacrylate, rayon, or any other suitable material or combinations thereof. In some embodiments, the absorbent element 596 can encircle the entire perimeter of the obturator 590. Alternatively, the absorbent element can be disposed at discrete locations along a perimeter of the obturator 590. In some embodiments, the absorbent element 596 can have a thickness of between about 0.5 mm and about 10 mm, including all values and sub-ranges therebetween.

FIG. 14 is a diagram of a liquid/gas interconnect, according to an embodiment. The liquid/gas interconnect 1437 can be structurally and/or functionally similar to other liquid/gas interconnects described herein, including, for example, liquid/gas interconnect 337. As shown, gas flow 1466 enters through a gas line 1462 and a valve 1464 and liquid flow 1476 enters through a liquid line 1472 and a valve 1474. The gas flow 1466 and the liquid flow 1476 merge and exit as a liquid/gas output 1483. The gas line 1462 and the liquid line 1472 can be structurally and/or functionally similar to other gas lines and liquid lines described herein, respectively, including, for example, gas line 262 and liquid line 272.

As described above, a cleaning system as described herein can be configured to use a pressurized gas (e.g., carbon dioxide) to propel a set volume of liquid into a trocar channel, e.g., to deliver it as a high-energy spray that can wash a distal end of an endoscope. The liquid/gas interconnect 1437, similar to other liquid/gas interconnects described herein, can be configured to combine the set volume of liquid with the pressurized gas to enable the delivery of the gas/liquid spray.

As depicted in FIG. 14, the gas flow 1466 designates a flow path of gas through the interconnect 1437. A valve 1464 regulates the flow of gas therethrough. In some embodiments, the valve 1464 can be controlled manually (e.g., the user can control when the valve 1464 is open and how much the valve 1464 is open). Alternatively, the valve 1464 can be configured to open automatically, e.g., when the pressure of the gas on the proximal side of the valve 1464 (i.e., the side of the valve 1464 coupled to the gas line 1462) exceeds a threshold value. In some embodiments, the threshold pressure to open the valve 1464 can be at least about 20 psi to at least about 50 psi, including all sub-ranges and values therebetween. For example, the threshold pressure to open the valve 1464 can be at least about 30 psi, at least about 35 psi, or at least about 40 psi. In some embodiments, the gas delivered by the gas source can include CO₂, nitrogen, argon, or any other inert gas or combinations thereof. In some embodiments, the valve 1464 can be a check valve. In some embodiments, the valve 1464 can prevent backflow of liquid into the gas line 1462.

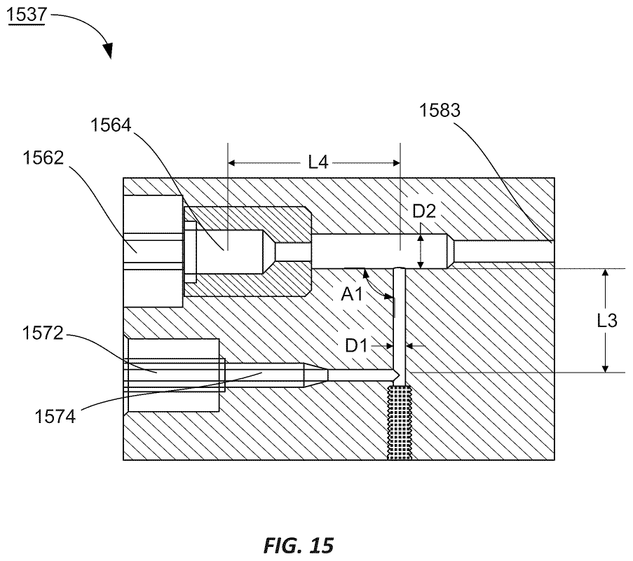

As depicted in FIG. 14, the liquid flow 1476 designates a flow path of liquid (e.g., wash solution) through the liquid/gas interconnect 1437. The valve 1474 regulates the flow of wash solution therethrough. The valve 1474 can be configured to prevent backflow of the liquid. The diameter of the liquid line 1472 (e.g., DI as shown in FIG. 15) can be sized to prevent bubble formation. In particular, the valve 1474 can prevent backflow of the liquid and the size of the liquid line 1474 can hold the liquid in place until the gas passes through the valve 1474 to draw out the liquid. In some embodiments, the valve 1474 can be controlled manually. In some embodiments, the valve 1474 can be configured to automatically open. For example, the opening and closing of the valve 1474 can be triggered when the pressure of the liquid on the proximal side of the valve 1474 (i.e., the side of the valve 1474 coupled to the liquid line 1472) exceeds a threshold value. In some embodiments, the threshold pressure to open the valve 1474 can be lower than the threshold valve to open the valve 1464 and/or valve 1484. As such, liquid can be delivered via the liquid line 1472 into an internal volume of the liquid/gas interconnect 1437 without leaking into the gas line and/or out through the output of the liquid/gas interconnect 1437. In use, liquid from a liquid supply (e.g., external liquid source 170, liquid reservoir 114) coupled to the liquid line 1472 can be delivered into an internal volume defined by various lumens of the liquid/gas interconnect 1437. The liquid that is delivered can have a set volume, e.g., for use in a wash sequence. The delivery of the liquid into the liquid/gas interconnect 1437 can be controlled by a controller (e.g., controller 220), as described above with reference to FIG. 2. For example, the controller can include a pump actuator (e.g., pump actuator 226) that is coupled to a pump mechanism 216, which can be used to pump a set volume of liquid into the liquid/gas interconnect 1437 in preparation for each wash sequence. In some embodiments, the controller can be configured to pump the set volume of liquid into the liquid/gas interconnect 1437 after each wash sequence in preparation for a subsequent wash sequence. In some embodiments, the controller can be configured to pump the set volume of liquid into the liquid/gas interconnect 1437 in response to a trigger event (e.g., the detection of an endoscope being withdrawn by a physician, which can be detected, for example, by a sensor). The process of delivering each set volume of liquid into the liquid/gas interconnect 1437 can be referred to as priming. In some embodiments, the valve 1474 can be a check valve. In some embodiments, the valve 1474 can prevent backflow of liquid and/or gas into the liquid line 1472.

The gas flow 1466 and the liquid flow 1476 can merge at a ‘T’ intersection. In use, liquid can be delivered via liquid line 1472 into the liquid/gas interconnect 1437 and held within the liquid/gas interconnect 1437 (e.g., in a space defined between valves 1464, 1474, 1484). When a wash sequence is triggered, e.g., due to a retraction of an endoscope, the gas line 462 can deliver a high-pressure gas into the liquid/gas interconnect 1437, which can force open valves 1464, 1484 and propel a predetermined volume or bolus of liquid out of the liquid/gas interconnect 1437. This liquid/gas output 1483 can then be delivered into a liquid/gas port of a trocar (e.g., trocar 330) and into the trocar channel (e.g., trocar channel 333) to wash an instrument (e.g., endoscope) disposed within the trocar channel. In some embodiments, each predetermined bolus of liquid can include at least about 1 μL, at least about 2 μL, at least about 3 μL, at least about 4 μL, at least about 5 μL, at least about 6 μL, at least about 7 μL, at least about 8 μL, at least about 9 μL, at least about 10 μL, at least about 11 μL, at least about 12 μL, at least about 13 μL, at least about 14 μL, at least about 15 μL, at least about 16 μL, at least about 17 μL, at least about 18 μL, or at least about 19 μL of liquid. In some embodiments, each bolus of liquid can include no more than about 20 μL, no more than about 19 μL, no more than about 18 μL, no more than about 17 μL, no more than about 16 μL, no more than about 15 μL, no more than about 14 μL, no more than about 13 μL, no more than about 12 μL, no more than about 11 μL, no more than about 10 μL, no more than about 9 μL, no more than about 8 μL, no more than about 7 μL, no more than about 6 μL, no more than about 5 μL, no more than about 4 μL, no more than about 3 μL, or no more than about 2 μL. Combinations of the above-referenced volumes for each bolus of liquid are also possible (e.g., at least about 1 μL and no more than about 20 μL or at least about 5 μL and no more than about 15 μL), inclusive of all values and ranges therebetween. In some embodiments, each bolus can include about 1 μL, about 2 μL, about 3 μL, about 4 μL, about 5 μL, about 6 μL, about 7 μL, about 8 μL, about 9 μL, about 10 μL, about 11 μL, about 12 μL, about 13 μL, about 14 μL, about 15 μL, about 16 μL, about 17 μL, about 18 μL, about 19 μL, or about 20 μL of liquid.

The bolus of liquid can be delivered over a time period of about 100 ms, about 110 ms, about 120 ms, about 130 ms, about 140 ms, about 150 ms, about 160 ms, about 170 ms, about 180 ms, about 190 ms, about 200 ms, about 210 ms, about 220 ms, about 230 ms, about 240 ms, about 250 ms, about 260 ms, about 270 ms, about 280 ms, about 290 ms, or about 300 ms, inclusive of all values and ranges therebetween. In some embodiments, an optional valve 1484 can be disposed downstream from the ‘T’ connection point between the gas and liquid flow paths. The valve 1484 can be configured to allow a bolus of liquid therethrough when a threshold pressure is reached. In some embodiments, the valve 1484 can be a check valve. As such, the valve 1484 can be configured to allow the liquid/gas output 1483 to be delivered into a trocar while preventing backflow of any liquid or gas that may remain within a trocar after a was sequence. In some embodiments, the valve 1484 can be structurally and/or functionally similar to the valve 1464. In some embodiments, the liquid/gas interconnect 1437 may not include a valve 1484. In such embodiments, the Venturi effect from the diameter of the gas line 1462 relative to the diameter of the liquid line 1472 can hold the liquid in place until a blast of gas draws the liquid out of the liquid line 1472 and pushes the liquid out of the liquid/gas output 1483.

The passages or lumens for the flow of gas (e.g., gas flow 1466) and the flow of liquid (e.g., liquid flow 1476) can be configured to have dimensions and/or geometry that facilitates flow in a preferential direction (e.g., toward a trocar) while preventing or reducing the likelihood of flow in the opposite direction (e.g., away from a trocar). An example of such dimensions and/or geometry is described with reference to the interconnect depicted in FIG. 15. FIG. 15 is a detailed diagram of a liquid/gas interconnect 1537, according to an embodiment. As shown, the liquid/gas interconnect 1537 includes a gas line 1562, a valve 1564, a liquid line 1572, a valve 1574, and a liquid/gas output 1583. In some embodiments, the gas line 1562, the valve 1564, the liquid line 1572, the valve 1574, and the liquid/gas output 1583 can be the same or substantially similar to the gas line 162, the valve 1464, the liquid line 1472, the valve 1474, and the liquid/gas output 1483, as described above with reference to FIG. 14. Thus, certain aspects of the gas line 1562, the valve 1564, the liquid line 1572, the valve 1574, and the liquid/gas output 1583 are not described in greater detail herein.

As shown, the gas line 1562 and the liquid line 1572 form an intersection angle A1. In some embodiments, the intersection angle A1, can be about 60 degrees, about 65 degrees, about 70 degrees, about 75 degrees, about 80 degrees, about 85 degrees, about 90 degrees, about 95 degrees, about 100 degrees, about 105 degrees, about 110 degrees, about 115 degrees, or about 120 degrees, inclusive of all values and ranges therebetween. Desirably, the gas line 1562 and the liquid line 1572 can merge at a ‘T’ intersection or connection, i.e., where the intersection angle Al is about 90 degrees. A ‘T’ connection can optimize the Venturi effect on the flow of liquid as it merges with the flow of gas.

In some embodiments, the liquid/gas output 1583 can couple to a port (e.g., a liquid/gas port of a trocar) that is fluidically coupled to an ejection port of a trocar. As such, the combined stream of gas and liquid can be ejected from the ejection port and into a trocar channel, e.g., to clean an endoscope positioned within the channel. In some embodiments, the liquid/gas output 1583 can be threaded, e.g., to facilitate coupling to a valve, port, or other structure.

As noted above, the diameters, lengths, and/or other geometry of the lumens or passages within the liquid/gas interconnect 1537 can be selected to allow a predetermined volume of fluid to be delivered to the trocar during each wash sequence. For example, the inner diameter D1 of the section of the liquid line 1572 adjacent to the liquid/gas intersection, the inner diameter D2 of the gas line before and after the liquid/gas intersection, the length L1 of the section of the liquid line 1572 adjacent to the liquid/gas intersection, and/or the distance L2 from the liquid/gas intersection and the valve 1564 can be selected to facilitate capture of a set volume of liquid from the liquid line during each timed release of the gas through the gas line 1562. As described above, gas can be delivered via gas line 1562 for a predetermined period of time, during which a predetermined volume of liquid is captured by the gas and propelled by the gas into the trocar lumen. After the wash sequence, the predetermined volume of liquid can then be re-filled into the liquid/gas interconnect 1537, e.g., during a priming sequence. As described above, the predetermined volume of liquid delivered during each wash sequence can be less than about 5 μL.

In some embodiments, D1 can be about 0.5 mm to about 1 mm, inclusive of all values and ranges therebetween. In some embodiments, D2 can be about 1 mm to about 2 mm, inclusive of all values and ranges therebetween. The ratio between D2 and D1 can be tuned to create a desired Venturi effect. For example, the ratio of D2 to D1 can be between about 1.5 and about 5, including, for example, about 2 and any other values of subranges therebetween. By having the liquid line have a smaller diameter than the gas line, liquid within the liquid line is less likely to leak into the gas line and gas within the gas line is less likely to enter the liquid line and form undesirable bubbles. When pressurized gas is released and travels through the gas line, it can pull the liquid within the liquid line out through the outlet of the interconnect.

As described with reference to FIG. 15, the valves 1564, 1574 can be selected to allow for flow of liquid and/or gas above certain pressures. The interconnect 1537 can define larger spaces for receiving the valves 1564, 1574, and then have those spaces taper down to the suitable dimensions of the lumens for enabling the precise delivery of the liquid during a wash sequence. The tapering of the gas line 1562 and/or the liquid line 1572 enables appropriate space to be provided for receiving the valves 1544, 1574 while still maintaining the appropriate diameters of the liquid and gas lines where the liquid and gas lines meet.

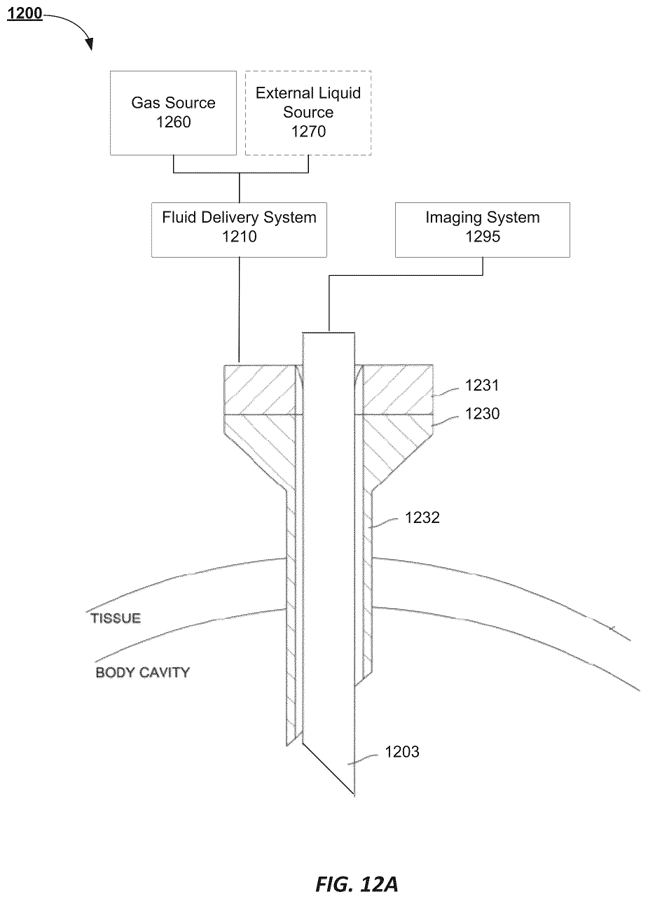

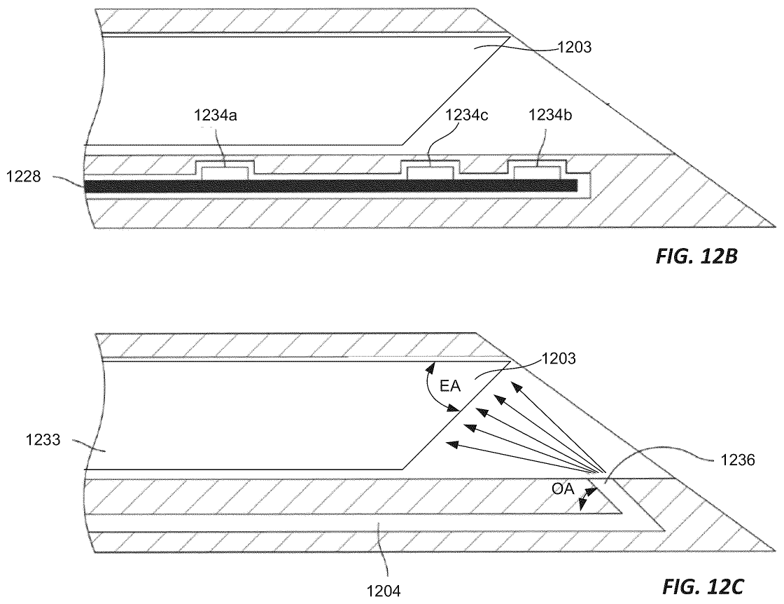

FIGS. 12A-12C show a trocar 1230 as part of a cleaning system 1200 when used with an endoscope 1203, according to embodiments. As shown, the trocar 1230 can be disposed within a body cavity that lies below a layer of tissue. The trocar 1230 includes a trocar hub 1231, a trocar shaft 1232, and a trocar channel 1233. The endoscope 1203 is coupled to an imaging system 1295. A fluid delivery system 1210, a gas source 1260, and an optional external liquid source 1270 are coupled to the trocar 1230. As depicted in FIG. 12B, an external circuit 1228 extends along the trocar 1230. The external circuit 1228 is electronically coupled to sensors 1234a, 1234b, 1234c (collectively referred to as sensors 1234). The trocar 1230 can also include a lumen 1204 that terminates at an orifice or ejection port 1236, e.g., where a liquid or wash solution can be ejected to wash the distal end of the endoscope 1203 positioned within the trocar channel. In some embodiments, the fluid delivery system 1210, the trocar 1230, the gas source 1260, and the external liquid source 1270 can be the same or substantially similar to the fluid delivery system 110, the trocar 130, the gas source 160, and the external liquid source 170, as described above with reference to FIG. 1. In some embodiments, the trocar hub 1231 and the trocar shaft 1232 can be the same or substantially similar to the trocar hub 331 and the trocar shaft 332, as described above with reference to FIG. 3. Thus, certain aspects of the fluid delivery system 1210, the trocar 1230, the trocar hub 1231, the trocar shaft 1232, the gas source 1260, and the external liquid source 1270 are not described in greater detail herein.

The imaging system 1295 can be any suitable imaging system that can be used with endoscopes or other imaging devices as described herein. In some embodiments, the imaging system 1295 can include processing circuitry, memory, and a user interface, e.g., for processing image data captured by the endoscope 1203 and displaying image data to a user (e.g., via the user interface). The endoscope 1203 can include a shaft with a lens and/or illumination system, e.g., for capturing image data within a body lumen or cavity. In some embodiments, the endoscope 1203 can include any of the features described in U.S. Patent Publication No. 2021/0127963.

As an illustrative example, the sensors 1234 may be configured to detect a position and/or orientation of the endoscope 1203. For example, sensor 1234a may be configured as a “scope present” sensor, and be configured to detect when the endoscope 1203 has been positioned within the trocar channel. Sensor 1234b may be configured as a “prime” sensor, and be configured to detect when the endoscope 1203 is being retracted within the trocar channel and therefore being positioned for a wash sequence. Detection of the endoscope 1203 may trigger a priming of the wash solution channel, e.g., such that the predetermined volume of liquid is positioned for ejection into the trocar channel. Sensor 1234c may be configured as a “wash” sensor, and be configured to detect when the endoscope 1203 has been positioned for cleaning or washing. For example, sensor 1234c may be configured to detect when the distal end of the endoscope 1203 is near the ejection port 1236 (e.g., slightly proximal of the ejection port 1236) such that a liquid/gas spray is delivered into the trocar channel to clean the distal end of the endoscope 1203. The electrical circuit 1228 may comprise a circuit board such as a flexible circuit board. Other circuits and electrical pathways may be used.

Alternatively, in some embodiments, fewer number of sensors may be used. For example, in some embodiments, the sensors 1234 may not include a prime sensor. In particular, a first sensor, e.g., sensor 1234c, can be disposed near the ejection port 1236 and be configured to send a signal to a controller (e.g., controller 220) when the distal end of the endoscope 1203 is near the ejection port 1236 and positioned for cleaning. Optionally, a second sensor, e.g., sensor 1234a, can also be disposed along the trocar channel at a position that is proximal of the first sensor. The second sensor can be configured to detect when the endoscope 1203 is initially being inserted into the trocar channel, and to send a signal to the controller to not initiate a wash sequence during the initial insertion of the endoscope 1203. The controller may be configured to trigger priming of the liquid line automatically after each wash sequence, thereby not requiring a separate prime sensor.

Still alternatively, in some embodiments, a greater number of sensors may be used. For example, additional sensors can be used to detect an orientation of the endoscope 1203, e.g., for when the endoscope 1203 has an angled distal end, as depicted in FIGS. 12B and 12C. With an angled distal end, the endoscope 1203 may need to be positioned such that the angled surface of the endoscope 1203 faces the ejection port 1236, e.g., for cleaning of the angled surface. Therefore, the endoscope 1203 may be rotated by a physician such that it is positioned to face the ejection port 1236. While the orientation of an angled endoscope is described herein, it can be appreciated that the orientation of the angled endoscope may not affect the cleaning of the endoscope. For example, sufficient pressures of liquid/gas spray may enable cleaning of the angled surface of the endoscope even when the angled surface of the endoscope does not face the ejection portion 1236. For example, pressures of between about 20 psi and about 50 psi (including all values and sub-ranges therebetween) may be used to cause sufficient spraying of the liquid/gas into the trocar channel to clean the surface of any type of endoscope, including, for example, flat and angled endoscopes.

As described above, the operation of the cleaning system may comprise priming of the system with a fixed or predetermined quantity of solution (e.g., about 1 μl to about 20 μl). In some embodiments, the priming can occur when a prime sensor detects the presence of the endoscope 1203 within the trocar channel. Alternatively, the priming can occur after delivery of liquid/gas during a wash sequence, e.g., in anticipation of a subsequent wash sequence. When the wash sequence is triggered, e.g., via sensor 1234C, pressurized gas may be activated for a fixed time period (e.g., about 100 ms to about 500 ms, including intervening end points such as about 200 ms and the like). The gas may serve one or more purposes, for example, as a propellant to atomize the solution into a high-energy spray as it exits the orifice (e.g., which may last about 100 ms to about 500 ms, including intervening endpoints) and/or as a drying system to remove excess solution from the distal end of the endoscope 1203 (e.g., which may last about 100 ms to about 500 ms, including intervening endpoints). In the latter case, the pressurized gas may be delivered alone without liquid. Other sequences may be used.

As shown, the orifice or ejection port 1236 is angled in a proximal direction at an orifice angle OA. In some embodiments, the distal surface of the endoscope 1203 can also angled, e.g., at an endoscope angle EA. As described above, when the endoscope 1203 has an angled distal end, it can be desirable to orient the endoscope such that the angled surface of the endoscope 1203 faces the ejection port 1236. This configuration allows for a substantial amount of the surface of the endoscope 1203 to be contacted by the wash solution or liquid spray exiting the ejection port 1236. In some embodiments, the orifice angle OA can be between about 5 degrees to about 90 degrees, inclusive of all values and sub-ranges therebetween.

In some embodiments, cleaning systems as described herein can be used to clean multiple different kinds and/or sizes of endoscopes. For example, a trocar (e.g., trocar 330, 1230) can have a trocar channel that is designed to receive endoscopes having an outer diameter of up to a predetermined value (e.g., up to about 10 mm). Endoscopes then having diameters that fit within the trocar channel can be cleaned using the systems and methods described herein, regardless of the size and/or configuration of the endoscopes. For example, a trocar channel large enough to receive a 10 mm endoscope can be configured to clean any endoscope that is smaller than or equal to 10 mm, including for example, a 5 mm or an 8 mm endoscope. The cleaning systems can also be designed to clean endoscopes having different distal tip configurations, including, for example, different angles, curvature, etc. In particular, the cleaning system can be designed to clean endoscopes have different angles, including, for example, 0, 30, or 45 degree angled lenses. To achieve this, the cleaning systems described herein can be configured to deliver a spray of liquid and/or gas at predetermined pressures above a threshold value that allows for sufficient cleaning of any shape or configuration of endoscope. In some embodiments, this threshold value can be between at least about 20 psi and about 50 psi, including, for example, at least about 35 psi.

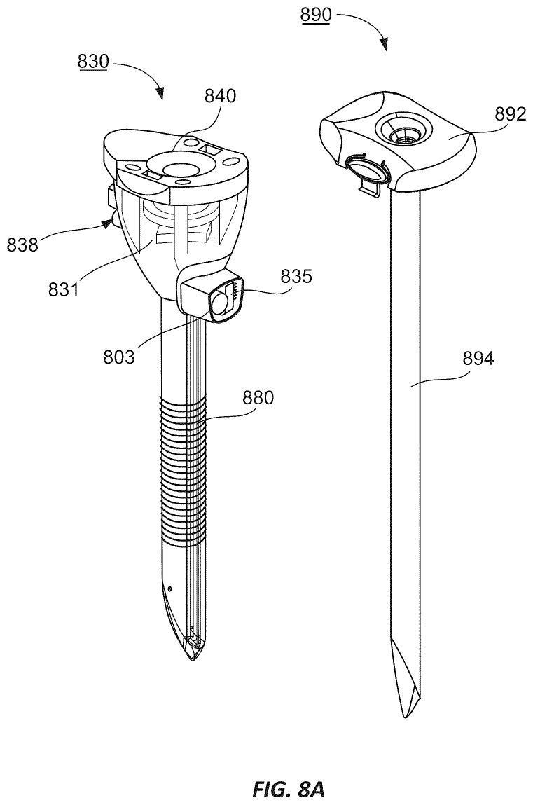

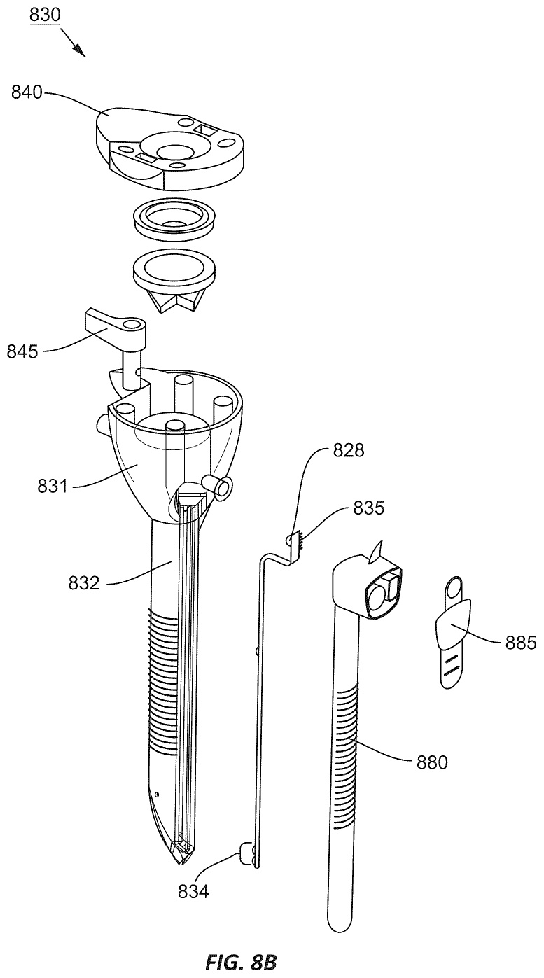

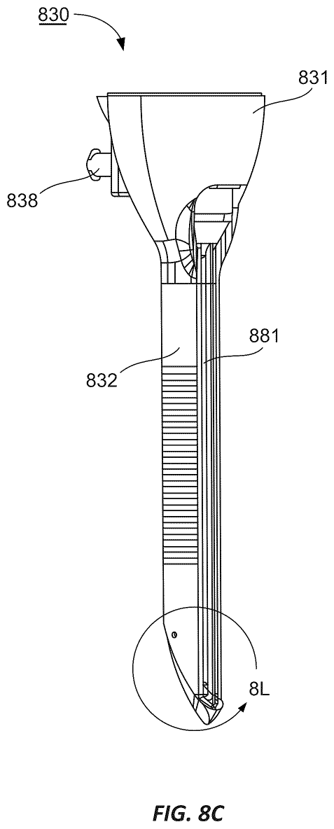

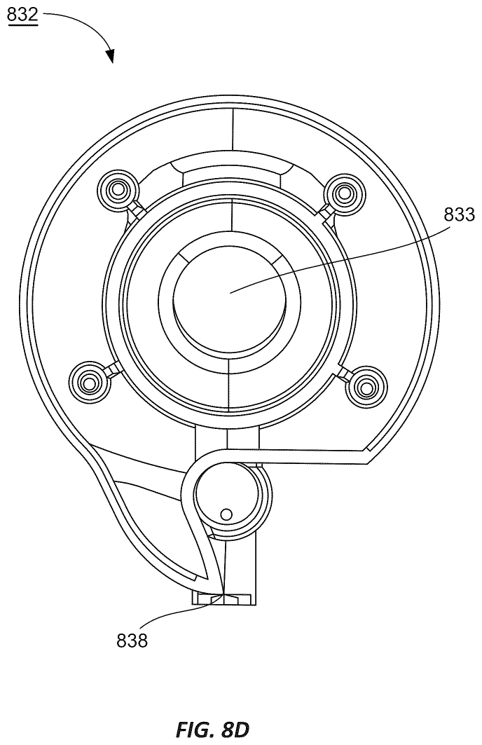

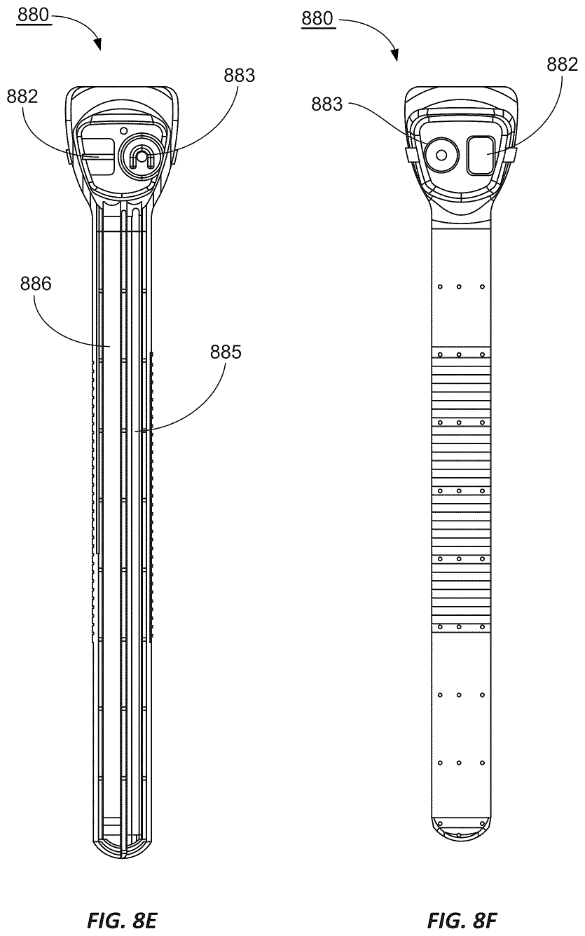

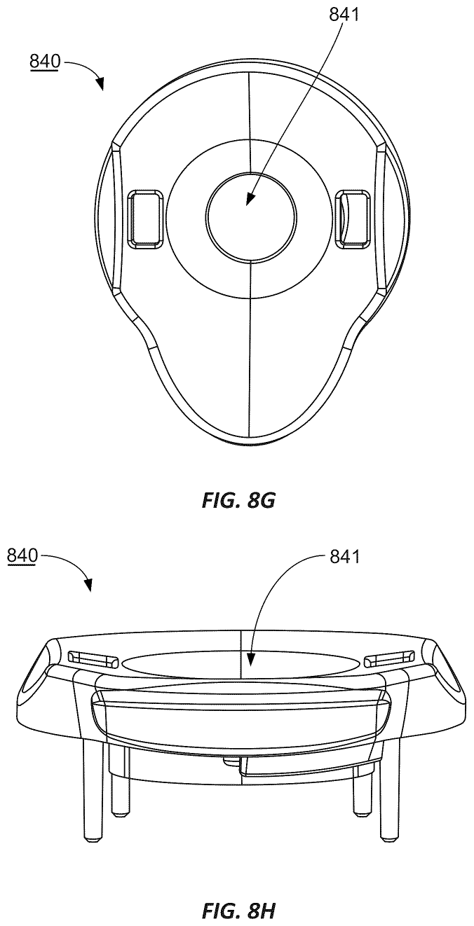

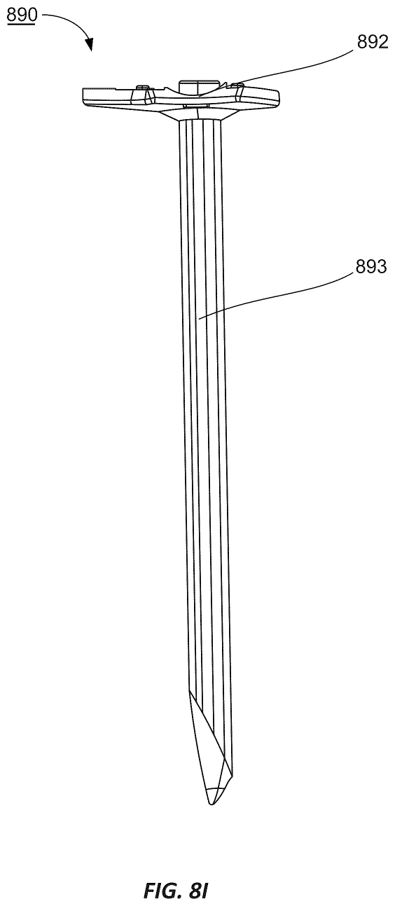

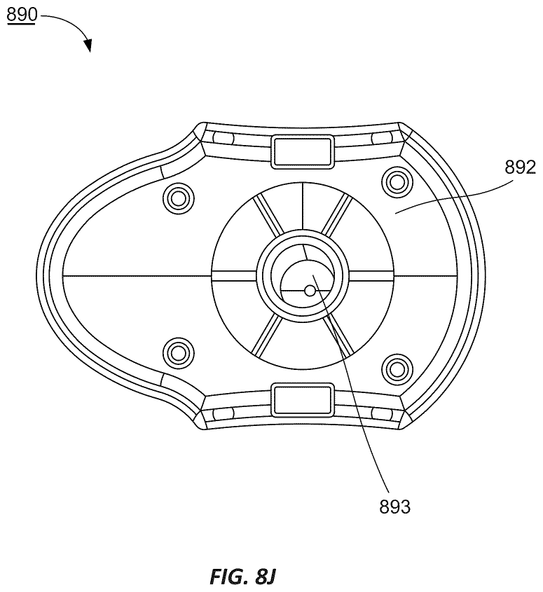

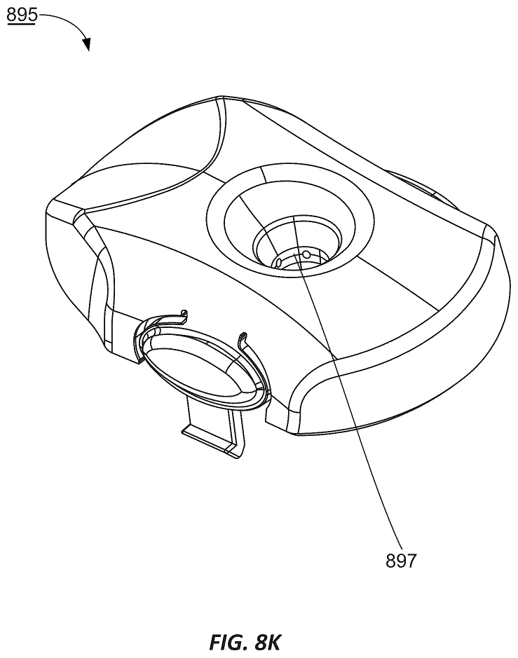

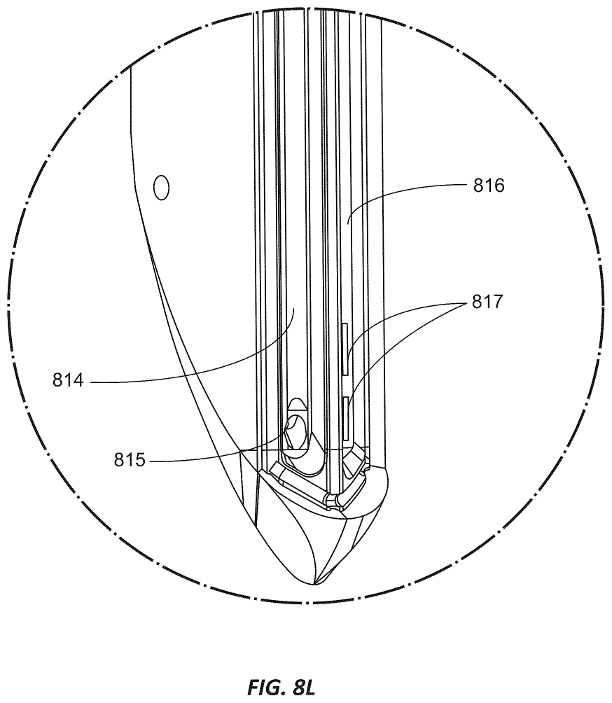

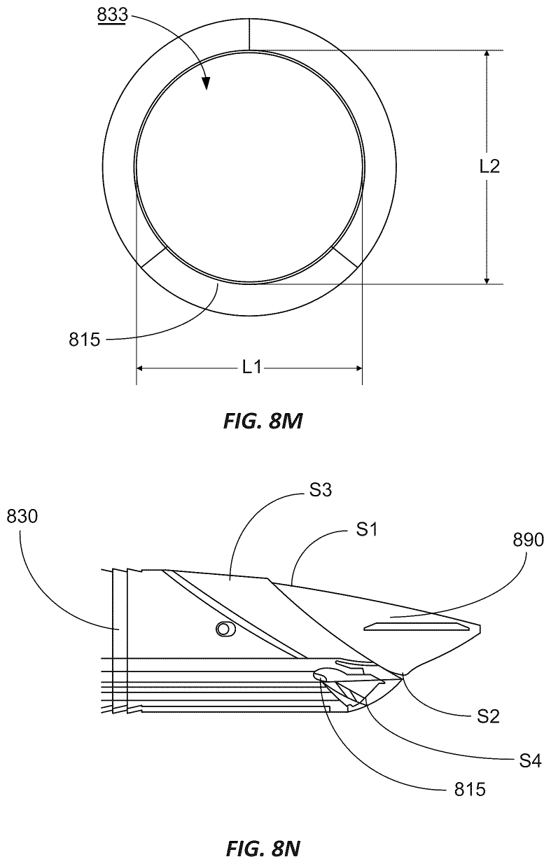

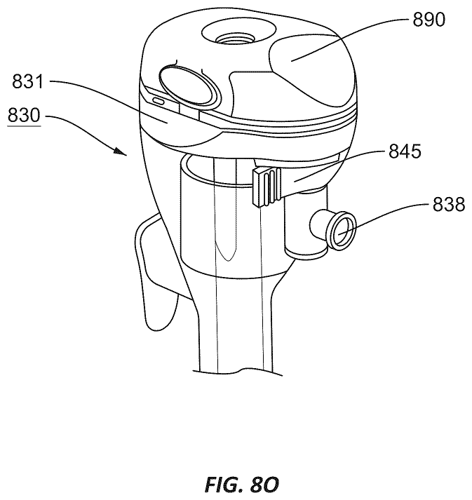

FIGS. 8A-8O are illustrations of a trocar 830 and an obturator 890 of a cleaning system, and various components thereof, according to embodiments. As shown, the trocar 830 includes a gas/liquid port 803, grooves 814, 816, an ejection port 815, openings or transparent sections 817, a PCB 828, a trocar hub 831, a trocar shaft 832, a trocar channel 833, sensor(s) 834, an electronic port 835, a vent 838, a trocar cover 840, a cover hole 841, a stopcock switch 845, a cap 880, cap grooves 885, 886, electronic port opening 882, and liquid/gas port 883. As shown, the obturator 890 includes an obturator hub 892, an obturator channel 893, an obturator cap 895, and an obturator cap hole 897. In some embodiments, the trocar 830, the trocar hub 831, the trocar shaft 832, the trocar channel 833, the sensor(s) 834, the electronic port 835, and the vent 838 can be the same or substantially similar to the trocar 330, the trocar hub 331, the trocar shaft 332, the trocar channel 333, the sensor(s) 334, the electronic port 335, and the vent 338 as described above with reference to FIG. 3. In some embodiments, the obturator 890, the obturator hub 892, and the obturator channel 893, can be the same or substantially similar to the obturator 590, the obturator hub 592, and the obturator channel 593, as described above with reference to FIG. 5. Thus, certain aspects of the trocar 830, the trocar hub 831, the trocar shaft 832, the trocar channel 833, the sensors 834, the electronic port 835, the obturator 890, the obturator hub 892, and the obturator channel 893 are not described in greater detail herein.

FIG. 8A shows a side-by-side view of the trocar 830 and the obturator 890. FIG. 8B shows an exploded view of the trocar 830. FIG. 8C shows a side view of the trocar 830 with a circular section indicating the perspective of FIG. 8L. FIG. 8D shows an overhead view of a trocar shaft 832. FIGS. 8E and 8F show a front and back view of the cap 880. FIG. 8G shows a top view of the trocar cover 840, while FIG. 8H shows a front view of the trocar cover 840. FIG. 8I shows a side view of the obturator 890, while FIG. 8J shows a top view of the obturator 890. FIG. 8K shows an auxiliary view of the obturator cap 895. FIG. 8L shows a detailed view of the bottom of the trocar 830. FIG. 8M shows a cross-sectional view of a trocar channel 833. FIG. 8N shows a bottom of the obturator 890 inserted into the trocar 830. FIG. 8O shows a detailed view of the trocar hub 831 with the obturator 890 disposed therein.

As shown, the PCB 828 can function as an electrical line that connects the sensor(s) 834 to the electronic port 835. In some embodiments, the sensor(s) 834 and the electronic port 835 can be disposed on the PCB 828. The electronic port 835 can be configured to be disposed within the electronic port opening 882, such that an electrical connection can be established between the electronic port 835 and an external electrical line (e.g., electrical line 282) via the electronic port opening 882. As described with reference to FIG. 10C, a trocar connection of a connector can be configured to couple to the trocar and, when coupled, an external electrical line housed within the connector can be configured to couple to the electronic port 835. The PCB 828 can be disposed within a passage that is collectively defined by the groove 886 of the cap and the groove 816 of the trocar shaft. In particular, the cap 880 can be configured to couple to the trocar shaft 832, as shown in FIG. 8A. The cap 880 can sit within a groove 881 defined along a side of the trocar shaft 832, and when coupled together, the groove 886 of the cap and the groove 816 of the trocar shaft can come together to form a passage for housing the PCB 828. This passage can be configured to maintain the PCB 828 in place, while also providing protection for PCB 828, e.g., by sealing PCB 828 within the passage such that fluids within the trocar channel and/or external to the trocar do not contact the PC 828.

As noted, one or more sensors 834 can be disposed on the PCB 828 toward a bottom or distal end of the PCB 828. In FIG. 8B, two sensors 834 are disposed at the distal end of the PCB 828, but it can be appreciated that a single sensor or more than two sensors can be disposed on the PCB 828. In an embodiment, the sensor(s) 834 can be light sensors. The light sensors can be disposed behind transparent portions 817 (or within openings 817) disposed along the groove 816 of the trocar. As such, the light sensors can be configured to detect when there is light within the trocar channel, e.g., as an indication of when a distal end of an endoscope may be near the sensors. In particular, when the trocar 830 is used for cleaning an endoscope, the endoscope can be disposed within the trocar channel of the trocar 830, and one or more light sensors can be disposed at a location near an ejection port 815 of the trocar 830. In particular, at least one light source can be disposed at the same point along the longitudinal length of the trocar as the ejection port 815. The distal end of the endoscope can emit a light, e.g., for providing illumination within a body cavity for capturing image data. When the endoscope is withdrawn or retracted within the trocar channel, e.g., to initiate a wash sequence, the light emitted by the endoscope may be detected by the light sensor(s). In some embodiments, the light sensor(s) can be configured to detect when a level of light within the trocar channel is greater than a predetermined threshold or has changed by a predetermined amount or percentage. In response to there being sufficient light (e.g., the level of light being above the threshold), which can be indicative of the position of the distal end of the endoscope being in close proximity to the ejection port, the cleaning system (e.g., via a controller such as, for example, controller 120, 220) can cause a volume of liquid and/or gas to be ejected from the ejection port 815 to clean the distal end of the endoscope. In some embodiments, the light sensor(s) can provide sensor data indicative of the level of light within the trocar channel, and a controller (e.g., controller 120, 220) of the cleaning system can be configured to determine when that level of light is above a threshold for ejecting the liquid volume. Alternatively, the light sensor(s) can be triggered to send a signal to a controller of the cleaning system when the level of light within the trocar channel is above a threshold. While this example cleaning operation is provided herein with reference to light sensors, it can be appreciated that other types of sensor(s) can also be used with the trocar cleaning systems as described herein, including, for example, pressure sensors, motion sensors, etc.

As depicted in the detailed view of FIG. 8L, at least one sensor 834 is configured to be disposed adjacent to the ejection port 815. By having at least one sensor 834 be close to the ejection port, the sensor 834 can provide a more accurate approximation of when the distal end of an endoscope may be in proximity to the ejection port 815. In some embodiments, the ejection port 815 can be angled retrograde, e.g., as depicted in FIG. 12C, and in such cases, the liquid volume can be set to be ejected when the distal end of the endoscope is proximal of the ejection port. Such positioning can enable the spray pattern as shown in FIG. 12C.

As further depicted in FIG. 8L, the ejection port 815 can be disposed at a distal end of a groove 814 that is defined along the trocar shaft or body. The groove 814 together with a groove 885 defined in the cap 880 can be configured to define a passage for delivering liquid and/or gas to the ejection port 815. In particular, the cap 880 as shown in FIGS. 8E and 8F defines two grooves. The first groove 886 for mating with the groove 816 to form the passage for housing the PCB 828, as described above. And a second groove 885 for mating with groove 814 to form a passage for delivering liquid and/or gas to the ejection port 815. When the cap 880 is coupled to the trocar shaft, the grooves 885, 814 can combine together to form the passage for delivering liquid and/or gas to the ejection port 815. Alternatively, in some embodiments, the cap 880 alone or the trocar shaft 832 alone can define the passage for delivering liquid and/or gas to the ejection port 815. The cap can include a liquid/gas port 883 that can be configured to connect to an output of a liquid/gas interconnect (e.g., the liquid/gas interconnect, as described above with reference to FIG. 3) for receiving volumes of liquid and/or gas. In some embodiments, the ejection port can have a diameter of between about 0.01 inches and about 0.5 inches, including all values and sub-ranges therebetween, including, for example, about 0.05 inches. In some embodiments, the ejection port 815 can be angled retrograde, e.g., similar to the ejection port 1236 described with reference to FIG. 12C. In some embodiments, the ejection port 836 can be angled from a longitudinal axis of the passage or grooves 885, 814 by between about 10 degrees and about 90degrees, including all values and sub-ranges therebetween (e.g., about 45 degrees, or between about 30 degrees and about 60 degrees). In some embodiments, the ejection port 836 can also be angled laterally (e.g., within a lateral plane of the trocar 830) or rotated off center from longitudinal plan of the passage or grooves 885, 814. For example, the ejection port 836 can be angled outward toward a side of the trocar channel by about 1 degree to about 20 degrees, including all values and sub-ranges therebetween (e.g., about 7.5 degrees rotated off center, or between about 5 degrees and about 10 degrees rotated off center). Alternatively, the ejection port 836 can be angled inward toward a centerline of the trocar channel by about 1 degree to about 20 degrees, including all values and sub-ranges therebetween.

As depicted in FIG. 8N, the passages for housing the PCB 828 and delivering fluid and/or gas can be disposed along a side S4 of the trocar shaft 832 that extends more distally than other sides of the trocar shaft 832. In particular, the ejection port 815 can be disposed along the more distally extending side S4 at a location that is distal to the distal end of the opposite side S3. As such, volumes of liquid and/or gas (e.g., liquid sprays) that are ejected by the ejection port 815 can spray generally toward a space that is not bounded on the opposite side by a wall of the trocar channel 833. Such positioning of the ejection port 815 can minimize or reduce entrapment or buildup of moisture within the trocar channel, thereby reducing the risk of fogging or condensation forming on the distal end of an endoscope during cleaning.

As noted above, the cap 880 can fit into the groove 881 on the trocar body or shaft 832. In some embodiments, the cap 880 can snap into place on the trocar shaft 832. In some embodiments, the cap 880 can be coupled to the trocar shaft 832 via an adhesive, a magnetic coupling, and/or a magnetic coupling.

The trocar 830 can include a stopcock switch 845 that can be rotated to open and close a vent 838. In some embodiments, the stopcock switch 845 can also be rotated between a first position for venting and a second position for insufflation. Further details of such a stopcock switch 845 are described with reference to FIGS. 9A-9C.

In some embodiments, the trocar shaft 832 and at least a portion of the trocar hub 831 can be formed as a unitary component, e.g., molded as a single piece. In some embodiments, the trocar hub 831 and the trocar shaft 832 can be transparent or semi-transparent or translucent. In some embodiments, the trocar hub 831 and trocar shaft 832 can be composed of a polymer, PVC, polypropylene, polyethylene, polycarbonate, or any other suitable material or combinations thereof. The trocar hub 831 can include a removable or attachable cover 840. The trocar cover 840 can attach to the trocar hub 831 via an interlocking mechanism, snap mechanism, or other mechanical coupling (e.g., fasteners, clips, etc.) Alternatively or additionally, the trocar cover 840 can attach to the trocar hub 831 via adhesive, magnetic coupling, etc. The trocar cover 840 includes the cover hole 841, e.g., for insertion of the obturator 890 and/or an endoscope.

The obturator 890 can fit into the trocar channel 833, e.g., to facilitate initial insertion of the trocar 830 into a body lumen or cavity. As shown in FIG. 8A, the obturator 890 includes a pointed distal end for piercing through body tissue. FIG. 8N depicts the distal end of the obturator 890 when it is fully inserted into the trocar channel 833. In such position, the obturator 890 can follow a profile of the trocar 830 at the distal end of the trocar 830. As shown, the obturator 890 can have a first angled surface S1 and a second angled surface S2. The first angled surface S1 and the second angled surface S2 can continue the respective angled surfaces of the trocar 830 and provide a continuous surface for penetrating through tissue. While not depicted in FIGS. 8A-8O, in some embodiments, the obturator 890 can include a wiping element and/or an absorbent element for cleaning the trocar channel 833 and/or an endoscope. As shown, the obturator 890 includes the obturator cap 895, which includes the hole 897 that can be used to receive an instrument (e.g., an endoscope). In some embodiments, a first endoscope having a smaller profile can be inserted within the obturator 890 during insertion of the trocar 830 into the body lumen or cavity, and then the obturator 890 and the first endoscope can be removed to allow for insertion of a second endoscope with a larger profile into the trocar channel. As shown in FIG. 8M, the trocar channel 833 can have an asymmetrical cross-section, similar to that described with reference to FIGS. 6B and 7. The asymmetrical cross-section of the trocar channel 833 can have a first lateral dimension L1 and a second lateral dimension L2, e.g., resulting from the overlap of two circular cross-sections. In some embodiments, the first lateral dimension L1 can be at least about 1 mm, at least about 2 mm, at least about 3 mm, at least about 4 mm, at least about 5 mm, at least about 6 mm, at least about 7 mm, at least about 8 mm, at least about 9 mm, at least about 10 mm, at least about 15 mm, or at least about 20 mm. In some embodiments, the first lateral dimension L1 can be no more than about 20 mm, no more than about 15 mm, no more than about 10 mm, no more than about 9 mm, no more than about 8 mm, no more than about 7 mm, no more than about 6 mm, no more than about 5 mm, no more than about 4 mm, or no more than about 3 mm. Combinations of the above-referenced values of the outer diameter L1 are also possible (e.g., at least about 2 mm and no more than about 20 mm or at least about 5 mm and no more than about 10 mm), inclusive of all values and ranges therebetween. The second lateral dimension L2 can be greater than the first lateral dimension L1. In particular, the second lateral dimension L2 can be at least about 1% to at least about 10% greater than the first lateral dimension L1, including all values and sub-ranges therebetween.

As described above with reference to FIGS. 6B and 7, the asymmetrical cross-section of the trocar channel 833 can be formed from two overlapping circular cross-sections. In use, an endoscope can be inserted through the larger of the two circular cross-sections and be held within that cross-section given its size and the small dimension smaller circular cross-section. The ejection port 815 can be disposed along perimeter of the smaller circular cross-section (see FIG. 8M) such that the ejection port 815 is spaced by a distance from the endoscope. Such positioning of the ejection port 815 can enable a larger spray to develop when liquid is ejected from the ejection port 815 and/or prevent closure or blocking of the ejection port 815 by the endoscope (e.g., in the case of a tight fit between the endoscope and the walls of the trocar channel 833).

The travel of liquid and/or gas from a liquid/gas interconnect through the trocar shaft 832 to the ejection port 815 can be frictionless or substantially frictionless. In particular, the trocar 830 (including trocar shaft 832 and cap 880) can be designed to provide a pathway for fluid flow that transitions from larger lumens down to a ultra-small ejection port in a frictionless manner, e.g., without sharp transitions (e.g., sharp turns or curves) and without sudden changes in passage diameter. Such can help prevent or reduce liquid trapping and/or bubble formation.

FIGS. 9A-9C show an example stopcock switch 945 with first and second passages 945a, 945b, according to an embodiment. In some embodiments, the stopcock switch 945 can be the same or substantially similar to the stopcock switch 845 described above with reference to the trocar 830. Thus, certain aspects of the stopcock switch 945 are not described in greater detail herein. The stopcock switch 945 can be integrated into a vent (not shown) and can be used to switch between coupling a first passage 945a or a second passage 945b to a vent hole of a trocar. The first passage 945a can be used for a first function (e.g., passive venting), while the second passage 945b can be used for a second function (e.g., insufflation). For example, when the stopcock switch 945 is in a first position, the first passage 945a can align with a vent hole or channel of a trocar, thereby allowing for passive venting. In some embodiments, the vent hole of the trocar can have a diameter of between about 1.0 mm and 3.0 mm, including for example, about 1.9 mm and other values and sub-ranges therebetween. In some embodiments, the diameter of the first passage 945a can be between about 1.0 mm and about 3.0 mm, including, for example, about 2.5 mm and other values and sub-ranges therebetween. When the vent hole of the trocar and the first passage 945a are aligned, they can collectively create a vent path that has a diameter equal to the lesser of the diameters of the vent hole of the trocar and the first passage 945a. For example, when a 2.5 mm inner diameter first passage 945a is aligned with a 1.9 mm inner diameter vent hole of a trocar, a 1.9 mm inner diameter vent path can be formed. The size of the vent path can be indicative of the venting rate, and adjustments to either the trocar vent hole or the first passage 945a can be made to provide for smaller and/or faster venting. In some embodiments, the first passage 945a and the second passage 945b can have the same diameter, while in other embodiments, the first passage 945a and the second passage 945b can have different diameters.

FIGS. 10A-10D are illustrations of various components of a wash or cleaning system, according to an embodiment. As shown, the system includes a trocar 1030, a connector 1078 including a controller connection 1084 (or proximal connection) and a trocar connection 1079 (or distal connection), a liquid/gas interconnect 1037 disposed in the trocar connection 1079, and a controller 1075. The trocar 1030 includes a gas/liquid port 1005, trocar hub 1031, a trocar shaft 1032, an electric port 1035, a vent 1038, and a stopcock switch 1045. The controller 1075 can be coupled to an electric wire 1076 and a gas line 1077. In some embodiments, the cleaning system depicted in FIGS. 10A-10D can be structurally and/or functionally similar to the cleaning systems described in other embodiments, and can include like components as those systems. For example, the connector 1078, the trocar 1030, the trocar hub 1031, the trocar shaft 1032, the liquid/gas interconnect 1037, and the controller 1075 can be the same or substantially similar to the connector 240, the trocar 330, the trocar hub 331, the trocar shaft 332, the liquid/gas interconnect 337, and the controller 120, 220, respectively, as described above with reference to FIGS. 1-3. In some embodiments, an obturator 1090 can be coupled to the trocar 1030, e.g., for facilitating placement of the trocar 1030 within the body lumen or cavity. The obturator 1090 can be the same or substantially similar to the obturator 590, as described above with reference to FIG. 5. Thus, certain aspects of the connector 1078, the trocar 1030, the trocar hub 1031, the trocar shaft 1032, the liquid/gas interconnect 1037, the obturator 1090, and the controller 1075 are not described in greater detail herein. FIG. 10A shows the trocar 1030, while FIG. 10B shows a detailed view of the trocar hub 1031 with the obturator 1090 disposed therein. FIG. 10C shows a coupling between the trocar 1030 and the trocar connection 1079. FIG. 10D shows a coupling between the controller 1075 and the controller connection 1084.

The controller 1075 can be operatively coupled to the trocar 1030, e.g., via connector 1078, and can control delivery of liquid and/or gas to the trocar 1030. The controller 1075 includes an outlet for connection to the controller connection 1084 of the connector 1078, which then connects to the trocar 1030. The controller 1075 is configured to receive power via the electrical wire 1076 and gas via the gas line 1077. In some embodiments, the gas line 1077 can be configured to deliver pressurized gas including CO₂, nitrogen, argon, or any other inert gas or combinations thereof.

The connector 1078 can include flexible tubing or insulation that houses an electrical line, a gas line, and/or a fluid line, as described with reference to FIG. 2. The connector 1078 includes the controller connection 1084 and the trocar connection 1079. The liquid/gas interconnect 1037 can be integrated into the trocar connection 1079. The trocar connection 1079 can be configured to couple to the trocar 1030, as shown in FIG. 10C. When the trocar connection 1079 and the trocar 1030 are coupled, an output of the liquid/gas interconnect 1037 can be coupled to a liquid/gas port disposed in the trocar and an electrical connector disposed within the trocar connection 1079 can be coupled to an electrical port 1035 in the trocar. As such, when the trocar connection 1079 is coupled to the trocar 1030, the connector 1078 can be configured to provide electrical and fluid communication between the trocar 1030 and the controller 1075. The controller connection 1084 can be configured to be coupled to the controller 1075. As shown in FIG. 10D, the controller connection 1084 can be received within a receptacle or opening within the controller 1075. In some embodiments, the controller connection 1084 can include an onboard liquid reservoir, e.g., liquid reservoir 214, that can be pumped (e.g., via a pump mechanism 216 and/or pump actuator 226 disposed within the controller 220) to deliver liquid to the liquid/gas interconnect 1037 for subsequent delivery into the trocar channel. Alternatively, the controller connection 1084 can include a port for coupling to a liquid reservoir disposed within the controller 1075 and/or coupled to the controller 1075, e.g., for receiving liquid for delivery to the liquid/gas interconnection 1037. The controller connection 1084 can also include a port for coupling to the gas source via gas line 1077 and a port or connector for coupling to the power source via power line 1076. The connector 1078, by having each of the electrical, gas, and liquid connections and lines, provides an efficient mechanism for coupling these lines between the controller 1075 and the trocar 1030. The connector 1078 can also be designed to house and protect the lines during operation, e.g., to reduce wear and/or tangling of the lines.

Additionally, the trocar 1030 can include a vent 1038 and a stopcock switch 1045. In some embodiments, the stopcock switch 1045 can be rotated to open and close the vent 1038. For example, the stopcock switch 1045 can be configured to rotate to close or open a passage through the vent 1038. In some embodiments, the stopcock switch 1045 can include multiple passages with different diameters, e.g., for different uses. For example, the stopcock switch 1045 can include a first passage with a first diameter for venting and a second passage with a second diameter for insufflation or venting at a different rate than the first passage. The stopcock switch 1045 can then be rotated to selectively position the passages for operation.

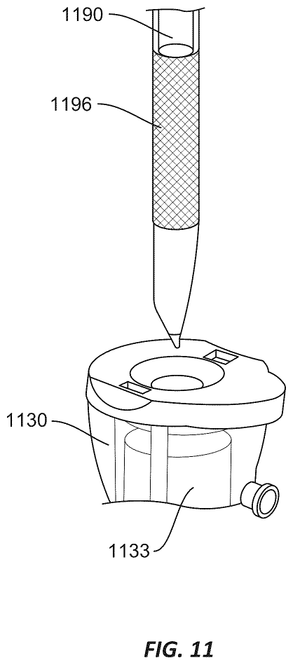

FIG. 11 shows an obturator 1190 with an absorbent element 1196 fitting into a trocar 1130. Upon insertion of the obturator into the trocar, the absorbent element 1196 can aid in cleaning the interior walls of a trocar channel 1133. In some embodiments, the absorbent element 1196 can include cloth, cotton, sponge, calcium chloride, silica gel, activated carbon, sodium polyacrylate, rayon, or any other suitable material or combinations thereof. As depicted in FIG. 11, the absorbent element 1196 can encircle the entire perimeter of the obturator 1190 and extend continuously for a set distance along a longitudinal axis of the obturator 1190. In some alternative embodiments, the absorbent element can be disposed on only a portion of the perimeter of the obturator 1190 and/or extend longitudinally at discrete points along the obturator 1190. In some embodiments, the trocar 1130 and the trocar channel 1133 can be the same or substantially similar to the trocar 330 and the trocar channel 333, as described above with reference to FIG. 3. In some embodiments, the obturator 1190 and the absorbent element 1196 can be the same or substantially similar to the obturator 590 and the absorbent element 596, as described above with reference to FIG. 5.

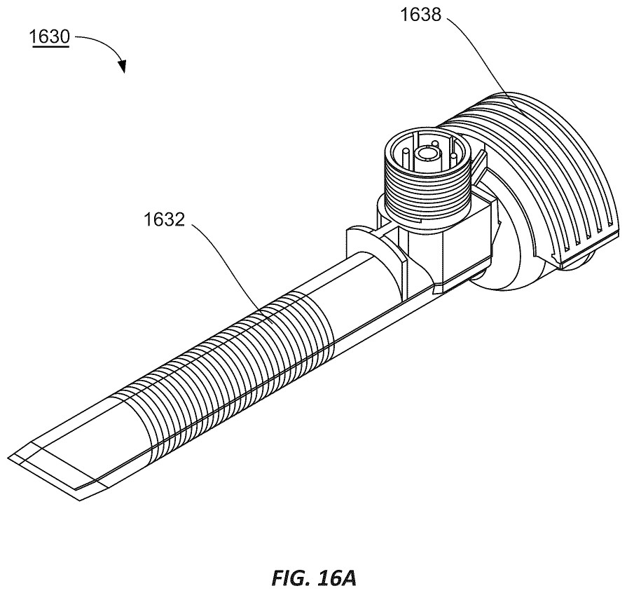

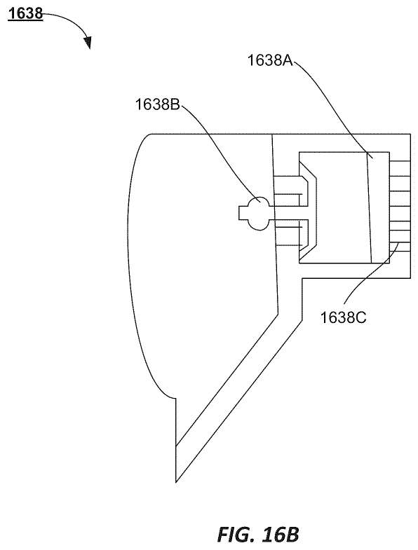

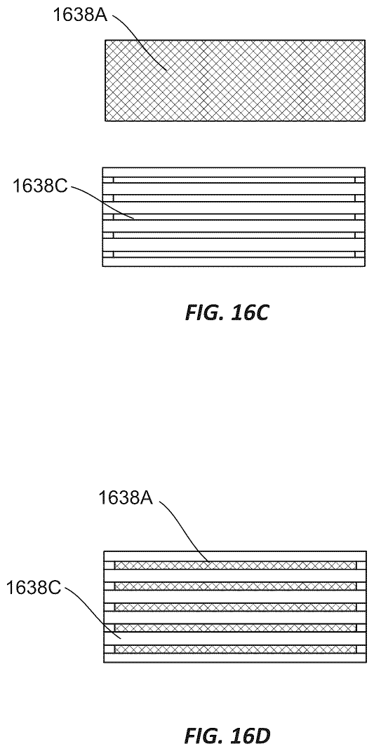

FIGS. 16A-16D show a trocar 1630 with venting and a filtration system, according to an embodiment. As shown, the trocar 1630 includes a trocar shaft 1632 and a vent 1638. The vent 1638 includes a filter 1638A, a valve 1638B, and a filter cover 1638C. In some embodiments, the trocar 1630 can be the same or substantially similar to other trocars described herein, including the trocar 330 as described above with reference to FIG. 3 and the trocar 830 as described above with reference to FIGS. 8A-80, and can include components that are structurally and/or functionally similar to those trocars. Thus, certain aspects of the trocar 1630 are not described in greater detail herein.

As described above, the vent 1638 is configured to vent gases from within the body lumen or cavity to an outside of the body lumen or cavity, e.g., to prevent pressure buildup within the body lumen or cavity. In some embodiments, the vent 1638 can be positioned such that the vented gases are directed away from electrical and/or fluid connections (e.g., directed away from a trocar connection of a connector such as, for example, connector 240) to prevent excessive humidity near electrical connections. In some embodiments, the vent 1638 can be configured to have standard size and/or geometry, e.g., to allow for the use of off-the-shelf filters with the vent. In some embodiments, the vent 1638 (or a port of the vent 1638) can be coupled to an active evacuation system (e.g., a smoke or gas evacuation system, such as, for example, an aspiration or suction system) to allow for active removal of gases from the body lumen or cavity. In some embodiments, the size of the vent 1638 can be modified for different surgery or patient types, e.g., to provide for different rates of venting and/or to facilitate maintaining a certain pressure within the body lumen or cavity. In some embodiments, the vent 1638 can vent gas from the abdominal cavity. In some embodiments, the vent 1638 can vent gas from the thoracic cavity when operating on the lungs. In some embodiments, the minimum diameter of the vent 1638 can be between about 1.0 mm and about 3.0 mm, including all values and sub-ranges therebetween. For example, in an embodiment, the minimum diameter of the vent 1638 can be at least about 1.6 mm, about 1.9 mm, about 2.1 mm, about 2.2 mm, or about 2.4 mm. Larger diameter vents can be associated with larger flow rates and venting.

The vent 1638 can include a filter 1638A along the evacuation path. The filter 1638A can capture particles within the vented gases, e.g., to reduce smells, contaminants, etc. within the vented gases. In some embodiments, the filter can include polyester, activated carbon, stainless steel, fiberglass, knitted mesh, polyethylene, foam, air filter, ceramic, polypropylene, or any other suitable material or combinations thereof. In some embodiments, the filter 1638A can be used with a filter cover 1638C. In some embodiments, the filter cover 1638C can prevent ejection of debris not captured by the filter 1638A. In some embodiments, the filter cover 1638C can attenuate noise. In some embodiments, the filter cover 1638C can prevent external debris (e.g., blood or other bodily fluid on physician's gloves) from contacting the filter 1638A. The vent 1638 can also include a valve 1638B (e.g., a butterfly valve or other suitable valve) along the evacuation path. The valve 1638B can be configured to allow for flow or venting when the pressure inside the trocar 1630 reaches a threshold value. The threshold value can be set to facilitate maintaining a certain level of pressure within the body lumen or cavity but to allow venting when such intraluminal pressure reaches higher levels. In some embodiments, the threshold pressure can be between about 5 mm Hg and about 100 mm Hg, including all values and sub-ranges therebetween. For example, the threshold valve can be set to 20 mm Hg, above which the valve 1638B would open and allow for venting.

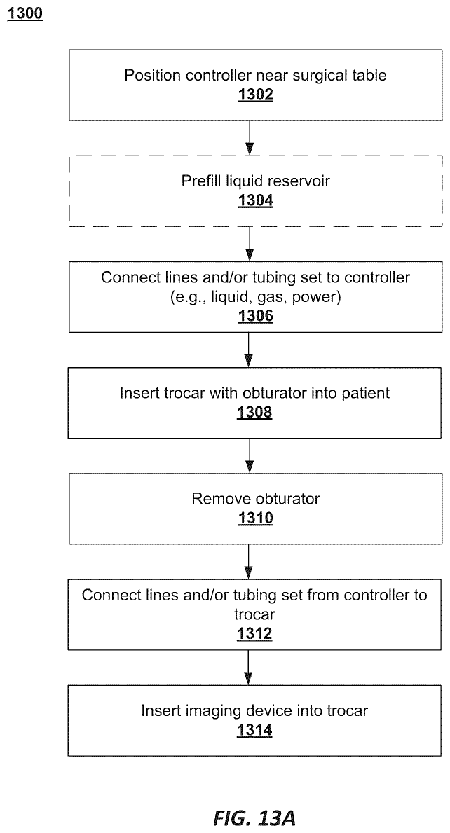

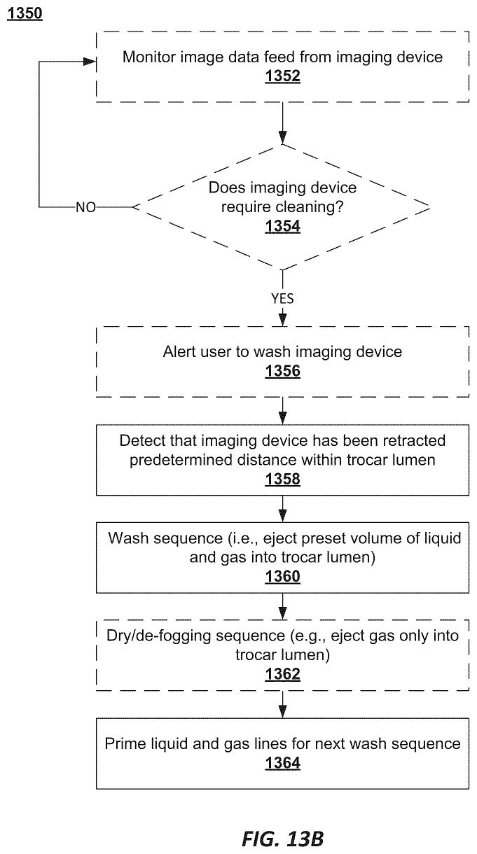

FIGS. 13A-13B illustrate methods associated with operating cleaning systems, according to various embodiments. FIG. 13A shows a method 1300 of setting up a cleaning system as described herein. As shown, the method 1300 includes positioning a controller (e.g., controller 120) near a surgical table at 1302. The method 1300 optionally includes prefilling a liquid reservoir (e.g., liquid reservoir 214) at 1304. The method 1300 further includes connecting lines and/or a tubing set to the controller at 1306, inserting a trocar (e.g., trocar 330) with an obturator (e.g., obturator 590) into a patient at 1308, removing the obturator at 1310, connecting lines (e.g., electric wire 1076) and/or a tubing set (e.g., gas line 1077) from the controller to the trocar at 1312, and inserting an imaging device (e.g., endoscope 1203) into a trocar at 1314.

In greater detail, at 1302, the controller is positioned near a surgical table. The controller, as described with reference to FIGS. 1 and 2, can control operation of the endoscope cleaning system. At 1304, a liquid reservoir, e.g., disposed within a controller connection of a connector (e.g. connector 240), can be pre-filled with a liquid. In some embodiments, the liquid reservoir can be filled with wash solution. In some embodiments, the wash solution can include a saline solution, a buffered solution, a bio-compatible surfactant, and/or any of the wash solutions described in U.S. Patent Publication No. 2021/0127963, incorporated above by reference.

At 1306, lines and/or a connector (e.g., a tubing set) are connected to the controller. For example, a controller connection of a connector including a liquid line, a gas line, and/or an electrical line can be coupled to the controller. The connector can have a second end (e.g., a trocar connection) that can coupled to a trocar, and therefore provide fluidic coupling and electrical communication between the controller and the trocar. The controller can also be fluidically coupled to a pressurized gas supply, e.g., via tubing. In some embodiments, the gas can include air, CO₂, nitrogen, argon, or any other inert gas or combinations thereof. In some embodiments, the controller can also be fluidically coupled to an external liquid reservoir or liquid source (e.g., external liquid source 170). Alternatively, in some embodiments, the connector may include an onboard reservoir (e.g., reservoir 114, 214) and therefore a separate coupling to an external liquid reservoir may not be required.

At 1308, the trocar and obturator are inserted into the patient. When being inserted into the patient, the obturator may include an endoscope that is positioned within the obturator channel (e.g., obturator channel 593). The obturator can include a sharp, penetrating tip that can cut through tissue to form a passage into the body lumen or cavity. An insufflation line can also be coupled to the trocar or obturator, whereby a gas can be pumped into the body lumen or cavity once the trocar has been placed within the body lumen or cavity. At 1310, after the trocar has been positioned within the body lumen or cavity, the obturator and insufflation line are removed from the trocar.

At 1312, the lines and/or the connector (e.g., a tubing set) are connected to the trocar. For example, a trocar connection of the connector can be coupled to a port disposed on the trocar. The trocar connection can include an electrical connection and a gas and/or liquid connection (e.g., an output of a liquid/gas interconnect), which can mate with an electrical port and a gas/liquid port disposed in the trocar, respectively. Once the connector and the trocar are coupled, the liquid reservoir and the gas source can be fluidically coupled to the trocar, such that the wash solution can be ejected from the trocar (e.g., via an ejection port). At 1314, the imaging device (e.g., an endoscope) is inserted into the trocar channel. The imaging device can then be positioned within the trocar for the duration of a surgical procedure.

The details of operating a cleaning system, e.g., once a trocar of the cleaning system has been positioned within the body cavity and an imaging device (e.g., endoscope) has been positioned within the trocar channel, are now described with reference to FIG. 13B. FIG. 13B depicts a method 1350 for washing the imaging device. At 1352, image data feed from the imaging device can optionally be monitored for fouling. At 1354, it can be determined whether the imaging device requires cleaning. If not, the imaging device continues to be monitored at 1352. If yes, a user (e.g., surgeon or medical assistant) can optionally be alerted to wash the imaging device at 1356. In some embodiments, a compute device (e.g., a compute device that is part of an imaging system such as, for example, imaging system 1295 and/or a compute device associated with the cleaning system such as, for example, the controller) can automatically detect fouling of the imaging device based on the image feed and trigger an alert or a wash sequence. The method 1350 further includes detecting that the trocar has been retracted a predetermined distance within the trocar lumen at 1358 and the initiation of a wash sequence at 1360. The method 1350 optionally includes a drying and/or de-fogging sequence at 1362. The method 1350 further includes priming the liquid and gas lines for the next wash sequence at 1364.

In greater detail, at 1352, the image data feed from the imaging device is monitored and a determination is made as to whether the image device requires cleaning, at 1354. In some embodiments, the determination of whether the image device requires cleaning can be made by the user. In some embodiments, the determination can be made automatically by a compute device (e.g., a controller such as, for example, controller 120, 220 and/or a processor associated with an imaging system). For example, light capture by the image device can be monitored and if the capture does not exceed a predetermined threshold, an alert can be generated and/or wash sequence initiated.

At 1356, the user may be alerted to wash the imaging device. At 1358, the controller of the cleaning system can detect that the imaging device has been retracted a predetermined distance within the trocar lumen, e.g., by a user after being alerted to wash the imaging device. Once the imaging device has been retracted by the predetermined distance, the wash sequence ensues at 1360. The detecting can be done using a sensor (e.g., sensor(s) 334) that is configured to monitor an amount of light within the trocar channel. The sensor can be configured to send a signal to the controller in response to detecting an amount of light that is greater than a predefined threshold. Alternatively, the sensor can be configured to send a stream of data to the controller, and the controller can be configured to determine when the sensor data is indicative of light being greater than a predefined threshold. The predefined threshold can be selected to capture when the imaging device is sufficiently close to an ejection port of the trocar (e.g., ejection port 336) to deliver a liquid and/or gas spray. For example, the predefined threshold of light can be representative of when the imaging device and therefore its illumination is sufficiently close to the sensor, which can be positioned next to the ejection port.

At 1360, the wash sequence ensues. The wash sequence includes ejecting a spray of liquid to clean the distal end of the imaging device. In some embodiments, the wash sequence can include ejecting a preset or predetermined volume of liquid into the trocar lumen. In some embodiments, the liquid can include the wash solution. Once the wash sequence is complete, an optional drying/de-fogging sequence can ensue at 1362. In some embodiments, the drying/defogging sequence can include ejecting gas into the trocar lumen without liquid. The gas can aid in drying liquid droplets off the imaging device. The gas can be free or substantially free of moisture content. In some embodiments, the ejection of gas can automatically following the ejection of a preset volume or bolus of liquid, e.g., because the pressurized gas is used to carry and push the liquid out of the ejection port first before ejecting out of the ejection port. At 1364, the liquid line can be primed for the next wash sequence. In other words, the liquid line is filled with wash solution.

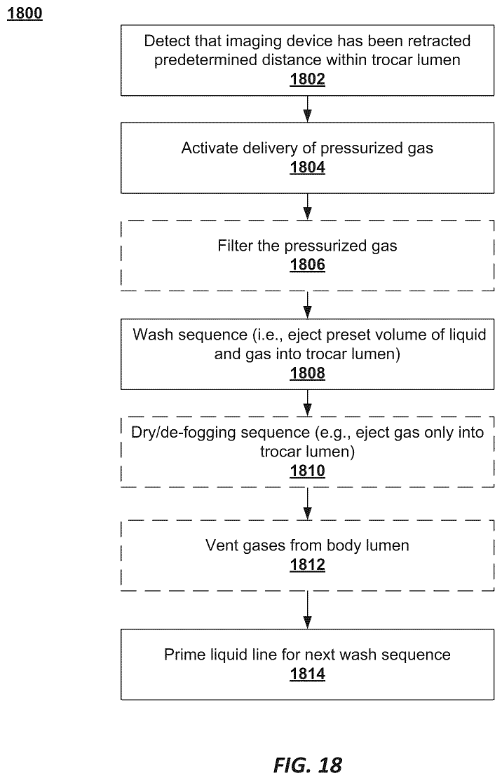

FIG. 18 illustrates a method 1800 associated with the operation of cleaning systems as described herein, according to various embodiments.

As described above, a trocar (e.g., trocar 330) can be positioned within a body lumen or cavity, e.g., with or without using an obturator (e.g., obturator 590). An imaging device such as an endoscope (e.g., endoscope 1203) can then be positioned within the trocar channel (e.g., trocar channel 333) for visualizing the inside of the body cavity. At 1802, the method 1800 includes detecting that the imaging device has been retracted a predetermined distance within the trocar channel. The detecting can be done using a sensor (e.g., sensor(s) 334) that is configured to monitor an amount of light within the trocar channel. The sensor can be configured to send a signal to a controller (e.g., controller 220) in response to detecting an amount of light that is greater than a predefined threshold. Alternatively, the sensor can be configured to send a stream of data to the controller, and the controller can be configured to determine when the sensor data is indicative of light being greater than a predefined threshold. The predefined threshold can be selected to capture when the imaging device is sufficiently close to an ejection port of the trocar (e.g., ejection port 336) to deliver a liquid and/or gas spray. For example, the predefined threshold of light can be representative of when the imaging device and therefore its illumination is sufficiently close to the sensor, which can be positioned next to the ejection port.

Once the imaging device has been retracted by the predetermined distance, the delivery of the pressurized gas can be activated, at 1804. Optionally, the pressurized gas being delivered can be filtered using a filter (e.g., filter 264).

The wash sequence ensues, at 1808. The wash sequence includes ejecting a spray of liquid to clean the distal end of the imaging device. In some embodiments, the wash sequence can include ejecting a preset or predetermined volume of liquid into the trocar lumen. In some embodiments, the liquid can include the wash solution. Once the wash sequence is complete, an optional drying/de-fogging sequence can ensue, at 1810. In some embodiments, the drying/defogging sequence can include ejecting additional gas into the trocar lumen without liquid. The gas can aid in drying liquid droplets off the imaging device. The gas can be free or substantially free of moisture content.

Optionally, at 1812, gases within the body lumen or cavity can be vented out of the body lumen or cavity, e.g., via a vent (e.g., vent 338). At 1814, the liquid line can be primed for the next wash sequence. In other words, the liquid line is filled with wash solution.

Various concepts may be embodied as one or more methods, of which at least one example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments. Put differently, it is to be understood that such features may not necessarily be limited to a particular order of execution, but rather, any number of threads, processes, services, servers, and/or the like that may execute serially, asynchronously, concurrently, in parallel, simultaneously, synchronously, and/or the like in a manner consistent with the disclosure. As such, some of these features may be mutually contradictory, in that they cannot be simultaneously present in a single embodiment. Similarly, some features are applicable to one aspect of the innovations, and inapplicable to others.

In addition, the disclosure may include other innovations not presently described. Applicant reserves all rights in such innovations, including the right to embodiment such innovations, file additional applications, continuations, continuations-in-part, divisionals, and/or the like thereof. As such, it should be understood that advantages, embodiments, examples, functional, features, logical, operational, organizational, structural, topological, and/or other aspects of the disclosure are not to be considered limitations on the disclosure as defined by the embodiments or limitations on equivalents to the embodiments. Depending on the particular desires and/or characteristics of an individual and/or enterprise user, database configuration and/or relational model, data type, data transmission and/or network framework, syntax structure, and/or the like, various embodiments of the technology disclosed herein may be implemented in a manner that enables a great deal of flexibility and customization as described herein.

All definitions, as defined and used herein, should be understood to control over dictionary definitions, definitions in documents incorporated by reference, and/or ordinary meanings of the defined terms.

As used herein, in particular embodiments, the terms “about” or “approximately” when preceding a numerical value indicates the value plus or minus a range of 10%. Where a range of values is provided, it is understood that each intervening value, to the tenth of the unit of the lower limit unless the context clearly dictates otherwise, between the upper and lower limit of that range and any other stated or intervening value in that stated range is encompassed within the disclosure. That the upper and lower limits of these smaller ranges can independently be included in the smaller ranges is also encompassed within the disclosure, subject to any specifically excluded limit in the stated range. Where the stated range includes one or both of the limits, ranges excluding either or both of those included limits are also included in the disclosure.

The phrase “and/or,” as used herein in the specification and in the embodiments, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the embodiments, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the embodiments, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the embodiments, shall have its ordinary meaning as used in the field of patent law.

As used herein in the specification and in the embodiments, the phrase “at least one,” in reference to a list of one or more elements, should be understood to mean at least one element selected from any one or more of the elements in the list of elements, but not necessarily including at least one of each and every clement specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the embodiments, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

As used in this specification, the singular forms “a,” “an,” and “the” include plural referents unless the context clearly dictates otherwise. Thus, for example, the term “a member” is intended to mean a single member or a combination of members, “a material” is intended to mean one or more materials, or a combination thereof.

The term “substantially” when used in connection with “cylindrical,” “linear,” and/or other geometric relationships is intended to convey that the structure so defined is nominally cylindrical, linear or the like. As one example, a portion of a support member that is described as being “substantially linear” is intended to convey that, although linearity of the portion is desirable, some non-linearity can occur in a “substantially linear” portion. Such non-linearity can result from manufacturing tolerances, or other practical considerations (such as, for example, the pressure or force applied to the support member). Thus, a geometric construction modified by the term “substantially” includes such geometric properties within a tolerance of plus or minus 5% of the stated geometric construction. For example, a “substantially linear” portion is a portion that defines an axis or center line that is within plus or minus 5% of being linear.

As used herein, the term “set” and “plurality” can refer to multiple features or a singular feature with multiple parts. For example, when referring to a set of devices, the set of devices can be considered as one device with multiple portions, or the set of devices can be considered as multiple, distinct devices. Thus, a set of portions or a plurality of portions may include multiple portions that are either continuous or discontinuous from each other. A plurality of particles or a plurality of materials can also be fabricated from multiple items that are produced separately and are later joined together (e.g., via mixing, an adhesive, or any suitable method).

While specific embodiments of the present disclosure have been outlined above, many alternatives, modifications, and variations will be apparent to those skilled in the art. Accordingly, the embodiments set forth herein are intended to be illustrative, not limiting. Various changes may be made without departing from the spirit and scope of the disclosure. Where methods and steps described above indicate certain events occurring in a certain order, those of ordinary skill in the art having the benefit of this disclosure would recognize that the ordering of certain steps may be modified and such modification are in accordance with the variations of the invention. Additionally, certain of the steps may be performed concurrently in a parallel process when possible, as well as performed sequentially as described above. The embodiments have been particularly shown and described, but it will be understood that various changes in form and details may be made.

1. An apparatus, comprising:
a shaft defining a channel for receiving an instrument, the shaft having a distal end that is disposable within patient anatomy;
an interconnector configured to couple to a liquid source and a gas source, the interconnector including a first valve configured to control delivery of the liquid and a second valve configured to control delivery of the gas, the interconnector configured to combine separate volumes of the liquid and the gas into a combined volume of the liquid and the gas;
an ejection port disposed near a distal end of the shaft, the ejection port fluidically coupled to the interconnector and configured to eject the combined volume of the liquid and the gas into the channel in response to a distal end of the instrument being disposed near the ejection port; and
a sensor disposed near the ejection port, the sensor configured to detect when the distal end of the instrument is disposed near the ejection port.

2. The apparatus of claim 1, wherein the interconnector further includes:
a liquid flow path configured to receive the liquid; and
a gas flow path configured to receive the gas,
the liquid flow path configured to intersect the gas flow path at a T-shaped connection.

3. The apparatus of claim 2, wherein the first valve and the second valve are disposed upstream of the T-shaped connection.

4. The apparatus of claim 2, wherein the volume of the liquid includes a predetermined volume of the liquid,
the first valve is configured to automatically open in response to a difference in pressure between an inlet and an outlet of the first valve to allow the predetermined volume of the liquid to flow into the liquid flow path.

5. The apparatus of claim 4, wherein the first valve is configured to prevent backflow of the predetermined volume of liquid through the first valve.

6. The apparatus of claim 4, wherein the volume of gas is a volume of high-pressure gas, and the second valve is configured to open to allow the volume of the high-pressure gas to flow through the gas flow path and propel the predetermined volume of the liquid into the channel.

7. The apparatus of claim 2, wherein a diameter of the gas flow path at the T-shaped connection is greater than a diameter of the liquid flow path at the T-shaped connection.

8. The apparatus of claim 1, wherein the shaft forms a part of a trocar, and
the interconnector is disposed in the trocar.

9. The apparatus of any one of claims 1-7, further comprising:
a controller configured to control delivery of the liquid and the gas to the interconnector; and
a cable configured to couple the controller to the shaft,
the interconnector being disposed in the cable.

10. The apparatus of claim 1, further comprising:
a controller operatively coupled to the sensor, the controller configured to:
receive, from the sensor, a signal indicative of the distal end of the instrument being disposed near the ejection port; and
in response to receiving the signal from the sensor, activate a wash sequence by triggering the delivery of the volume of the gas such that the volume of the gas mixes with and propels the volume of liquid into the channel.

11. The apparatus of claim 10, wherein the volume of the liquid is a first volume of the liquid, and the controller is further configured to:
control a pump mechanism to pump a second volume of liquid into the interconnector.

12. An apparatus, comprising:
a shaft defining a channel for receiving an instrument, the shaft having a distal end that is disposable within patient anatomy;
a cap couplable to the shaft, the cap including a fluid port configured to couple to a liquid source and a gas source and an electrical port configured to couple to a controller;
an ejection port disposed near a distal end of the shaft, the ejection port configured to eject a predetermined volume of liquid and gas into the channel in response to a distal end of the instrument being disposed near the ejection port; and
a fluid passage defined by at least one of the shaft or the cap and extending along a longitudinal length of the shaft, the fluid passage configured to convey the predetermined volume of liquid from the fluid port to the ejection port;
a sensor configured to detect when the distal end of the instrument is disposed near the ejection port; and
an electrical line disposed in at least one of the shaft or the cap and extending along the longitudinal length of the shaft, the electrical line configured to couple the electrical port to the sensor.

13. The apparatus of claim 12, wherein the channel has an asymmetric cross-section having a first lateral length that is greater than a second lateral length.

14. The apparatus of claim 12, further comprising:
a vent fluidically coupled to the channel and configured to vent gases from within the patient anatomy to an exterior of the patient anatomy.

15. The apparatus of claim 14, further comprising a stopcock switch, the stopcock switch configured to be rotated between first and second positions to open and to close the vent.

16. The apparatus of claim 12, wherein the electrical line includes a flexible printed circuit board.

17. The apparatus of claim 16, wherein the sensor is disposed on the flexible printed circuit board.

18. The apparatus of claim 12, wherein the shaft defines a groove, and the cap is configured to sit within the groove when coupled to the shaft.

19. An apparatus, comprising:
a shaft defining a channel for receiving an instrument, the shaft having a distal end that is disposable within patient anatomy;
an ejection port disposed near a distal end of the shaft, the ejection port configured to eject a predetermined volume of liquid and gas into the channel in response to a distal end of the instrument being disposed near the ejection port;
a sensor disposed near the ejection port, the sensor configured to detect when the distal end of the instrument is disposed near the ejection port;
a vent fluidically coupled to the channel and configured to vent gases from within the patient anatomy to an exterior of the patient anatomy; and
a filter disposed along a pathway of the vent and being configured to filter the gases being vented through the vent.

20. The apparatus of claim 19, further comprising a valve disposed along a pathway of the vent, the valve being configured to open the vent in response to pressure within the patient anatomy being greater than a predefined threshold.

21. The apparatus of claim 20, wherein the valve is configured to prevent flow of fluids external to the patient anatomy into the channel.

22. The apparatus of claim 20, wherein the filter is disposed downstream from the valve.

23. The apparatus of claim 19, further comprising a stopcock switch, the stopcock switch configured to be rotated between first and second positions to open and to close the vent.

24. The apparatus of claim 23, wherein the shaft is coupled to a hub, and the stopcock switch is disposed in the hub.

25. An apparatus, comprising:
a shaft defining a channel for receiving an instrument, the shaft having a distal end that is disposable within patient anatomy, the channel having an asymmetrical cross-section defined by a plurality of side walls where a first side wall of the plurality of side walls extends out further than one or more remaining side walls of the plurality of side walls;
an ejection port disposed near a distal end of the shaft, the ejection port configured to eject a predetermined volume of liquid and gas into the channel in response to a distal end of the instrument being disposed near the ejection port, the ejection port being located in the channel on the first side wall; and
a sensor disposed near the ejection port, the sensor configured to detect when the distal end of the instrument is disposed near the ejection port.

26. The apparatus of claim 25, wherein the asymmetrical cross-section has a shape corresponding to two overlapping circular cross-sections with offset centers, where a first circular cross-section of the two overlapping circular cross-sections has a larger diameter than a second circular cross-section of the two overlapping circular cross-sections.

27. The apparatus of claim 26, wherein the first circular cross-section is sized to receive the instrument such that the second circular cross-section provides a gap between the first side wall and an outer surface of the instrument when the instrument is received in the channel.

28. The apparatus of claim 25, wherein the sensor is located in the channel on the first side wall.

29. The apparatus of claim 25, wherein the ejection port and the sensor are disposed at the same position along a longitudinal length of the shaft.

30. The apparatus of claim 25, further comprising a vent fluidically coupled to the channel and configured to vent gases from within the patient anatomy to an exterior of the patient anatomy.
